(19) United States
(12) Patent Application Publication
(10) Pub. No.: US 2020/0362382 A1
(43) Pub. Date: Nov. 19, 2020

(54) METHODS OF PREPARING MODIFIED RNA

(71) Applicant: ModernaTX, Inc., Cambridge, MA (US)

(72) Inventors: Kristen OTT, Norwood, MA (US); Aaron LARSEN, Boston, MA (US); I-Ping CHAN, Newton, MA (US); Jennifer NELSON, Brookline, MA (US); William ISSA, Dedham, MA (US); Melissa J. MOORE, Chestnut Hill, MA (US)

(21) Appl. No.: 16/639,881

(22) PCT Filed: Aug. 17, 2018
(86) PCT No.: PCT/US2018/046933

§ 371 Date: Feb. 18, 2020

Related U.S. Application Data
(60) Provisional application No. 62/547,682, filed on Aug. 18, 2017.

(51) Int. Cl. C12P 19/34; C07H 21/02

(52) U.S. Cl. CPC C12P 19/34; C07H 21/02

(57) ABSTRACT

The present disclosure relates to methods for preparing a modified RNA comprising enzymatically ligating an acceptor moiety having a 3′-hydroxyl group to the 5′-end of a donor moiety (e.g., installation of 5′-triphosphate groups, mRNA caps, or cap-like structures on chemically synthesized RNA or installation of 5′-triphosphate groups, mRNA caps, cap-like structures, or non-cap like structures on enzymatically prepared RNA).

### RELATED APPLICATIONS

This application claims priority to, and the benefit of, U.S. Provisional Application No. 62/547,682, filed Aug. 18, 2017, the entire content of which is incorporated herein by reference in its entirety.

### INCORPORATION-BY-REFERENCE OF SEQUENCE LISTING

The contents of the text file named “MRNA040WOSL.txt”, which was created on Aug. 17, 2018, and is 1663 bytes in size, are incorporated herein by reference in its entirety.

### BACKGROUND

Solid-phase chemical synthesis of RNA allows for elaborate control over RNA modification by allowing modified nucleobases, sugars (or analogues), or backbones to be placed at virtually any location within an RNA with almost total control. This is in contrast to preparing modified RNA using in vitro transcription (IVT), a process that requires a canonical nucleotide triphosphate (NTP) to be entirely replaced with a modified NTP. This substitution results in either globally modified RNA or the stochastic incorporation of canonical and modified NTPs according to the ratio of modified to canonical NTPs present in the reaction and the substrate preferences of the polymerase.

The most critical limitation of the solid-phase chemical synthesis of RNA is the length of the resulting oligonucleotide. This length does not commonly exceed 100 nt at the extreme and is typically limited to around 60 nt. Due to the generally poor coupling efficiency of non-canonical amidites and the fact that some modifications require many chemical steps for installation, the length of chemically synthesized RNA is even more limited when certain modifications need be installed in the RNA. A particular difficulty is the installation of either a 5′-triphosphate or a 5′ cap-like structure (a structure similar to the 7-methylguanylate cap commonly found in naturally occurring mRNA). This is a special concern for mRNA therapeutics and vaccines as caps or cap-like structures are typically vital to translation (the biological process of producing proteins from mRNA). The presence of a 5′-triphosphate can be used to elaborate RNA into mRNA by the enzymatic addition of a cap or cap-like structure. Currently, the best processes for installing cap-like structures and 5′-triphosphate groups on mRNA are chemical methods performed in solid-phase (Thillier, Y. RNA 2012, 18, 856-868; Zlatev, I. Org. Lett. 2010, 12, 2190-2193). However, the oligonucleotides featured in these studies are typically very short, not exceeding 21 nt in length. These chemical methods and other similar methods are also time-consuming as they require very long reaction times and require the use of large quantities of expensive reagents.

Cap structures are typically featured at the 5′-end of mRNA and serve to: i) protect mRNA from nuclease degradation; ii) decrease innate immune response, and; iii) enable or enhance translational efficiency. Each feature is of significant use for mRNA therapeutics and/or mRNA vaccines.

The standard approach for the installation of mRNA caps is treating uncapped mRNA with the Vaccinia virus capping enzyme (VCE) in the presence of GTP and S-adenosylmethionine (SAM) in the appropriate buffer and at the appropriate temperature. This treatment results in the installation of a 7-methylguanylate cap structure at the 5′-end of the mRNA (Shuman, S., J. Biol. Chem. 1990, 265, 11960-11966). Subsequent or simultaneous treatment with mRNA Cap 2′-O-Methyltransferase adds a methyl group at the 2′-O position of the guanine immediately downstream of the 7-methylguanylate cap structure (Kuge, H. et al., Nucleic Acids Res. 1998, 26, 3208-3214). This capping system is highly specific in its activity and is generally understood to not be promiscuous with regards to modified substrates (ex. modified NTPs) that may result in the installation of modified mRNA caps. Modified mRNA caps may further improve protection of mRNA from nuclease degradation, decrease innate immune response, and improve translational efficiency. Each feature may be of significant use for mRNA therapeutics and/or mRNA vaccines. For this reason, an alternative and more promiscuous capping method is of value. Furthermore, mRNAs produced by IVT using T7 RNA polymerase generally begin with ‘G’ at the 5′-position. The nucleobase identity at this position is maintained when the RNA is capped using the VCE. For this reason, RNAs made in this way have very little sequence flexibility with regards to nucleobase identity at this position. The use of an alternative capping system may allow for the incorporation of other nucleobases at this position. This may be useful as many human mRNAs are not limited to ‘G’ at this position

### SUMMARY

The present disclosure relates to methods of preparing a modified RNA. In some embodiments, the methods of the disclosure include installation of 5′-triphosphate groups, mRNA caps (including modified mRNA caps) or cap like structures on RNA.

In some aspects, the present disclosure provides a method of preparing a modified RNA by enzymatically ligating an acceptor moiety to a donor moiety. In some embodiments, the present disclosure provides a method of preparing a modified RNA by enzymatically ligating an acceptor moiety to the 5′-end of a donor moiety. In some embodiments, the present disclosure provides a method of preparing a modified RNA by enzymatically ligating the 3′-end of an acceptor moiety to the 5′-end of a donor moiety. In some embodiments, the acceptor moiety has a 3′-hydroxyl group. In some embodiments, the donor moiety has a leaving group.

In further aspects, the present disclosure provides a modified RNA prepared by the methods disclosed herein. In some embodiments, the modified RNA is prepared by enzymatic ligation, i.e., enzymatically ligating an acceptor moiety to a donor moiety. In some embodiments, the modified RNA is prepared by enzymatically ligating an acceptor moiety to the 5′-end of a donor moiety. In some embodiments, the modified RNA is prepared by enzymatically ligating the 3′-end of an acceptor moiety to the 5′-end of a donor moiety. In some embodiments, the acceptor moiety has a 3′-hydroxyl group. In some embodiments, the donor moiety has a leaving group.

In some embodiments, this enzymatic ligation is performed in a single enzymatic step.

In some embodiments of the present disclosure, the method of the disclosure comprises the use of a ligation enzyme. In some embodiments, the ligation enzyme is a T4 DNA ligase. In some embodiments, the ligation enzyme is T4 RNA ligase 1. In some embodiments, the ligation enzyme is T4 RNA ligase 2. In some embodiments, the ligation enzyme is T3 DNA ligase. In some embodiments, the ligation enzyme is T7 DNA ligase.

In some embodiments of the present disclosure, the donor moiety is an RNA. In some embodiments, the donor moiety is an RNA comprising a non-naturally occurring nucleotide. In some embodiments of the present disclosure, the non-naturally occurring nucleotide comprises one or more chemical modifications of a naturally occurring nucleotide.

In some embodiments of the present disclosure, the naturally occurring nucleotide comprises chemical modifications that are located on the major groove face of the nucleobase portion of the nucleotide. In some embodiments, the chemical modification replaces an atom of the major groove face of the nucleobase with a group selected from optionally substituted amino, optionally substituted thiol, optionally substituted alkyl, optionally substituted alkenyl, and halo. In some embodiments, the chemical modification replaces an atom of the major groove face of the nucleobase with an amino. In some embodiments, the nucleobase portion of the non-naturally occurring nucleotide is a pyrimidine nucleobase.

In some embodiments of the present disclosure, the non-naturally occurring nucleotide comprises chemical modifications located on the sugar. In some embodiments, a modification is a modification at the 2′ position of the nucleoside. In some embodiments, the chemical modification is 2′-O alkylation. In some embodiments, the chemical modification is 2′-O methylation. In some embodiments, the chemical modification replaces an atom of the sugar with a group selected from optionally substituted amino, optionally substituted thiol, optionally substituted azido, optionally substituted alkyl, optionally substituted alkenyl, and halo.

In some embodiments of the present disclosure, the modification located on the sugar comprises a modification at the 4′ position of the nucleoside. In some embodiments, the chemical modification replaces an atom of the sugar with an a group selected from optionally substituted amino, optionally substituted thiol, optionally substituted azido, optionally substituted alkyl, optionally substituted alkenyl, and halo.

In some embodiments of the present disclosure, the non-naturally occurring nucleotide comprises chemical modifications located on the sugar and the sugar is modified at both the 2′ and 4′ positions of the nucleoside. In some embodiments, the chemical modification at the 2′ position is O-alkylation. In some embodiments, the chemical modification at the 2′ position is O-methylation. In some embodiments, the chemical modification at the 2′ or 4′ position replaces an atom of the sugar with a group selected from optionally substituted amino, optionally substituted thiol, optionally substituted azido, optionally substituted alkyl, optionally substituted alkenyl, and halo.

In some embodiments, the non-naturally occurring nucleotide comprises one or more chemical modifications located on the sugar-phosphate backbone. In some embodiments, the chemical modification on the sugar-phosphate backbone comprises replacing one or more oxygens of the phosphodiester linkage. In some embodiments one or more oxygens of the phosphodiester linkage are replaced with a group selected from amino, S, and BH₃. In some embodiments one or more oxygens of the phosphodiester linkage are replaced with a group selected from S and BH₃. In some embodiments, a hydroxyl of the phosphodiester linkage is replaced with a group selected from alkyl, alkoxy or hydrogen.

In some embodiments of the present disclosure, the donor RNA comprises one or more chemical modifications of the sugar and the internucleotide linkage of the donor RNA. In some embodiments, the donor RNA comprises a modification of the sugar at the 2′-position of the nucleoside. In some embodiments, the donor RNA comprises a modification of the sugar at the 4′-position of the nucleoside. In some embodiments, the donor RNA comprises a modification of the sugar at the 2′-position and the 4′-position of the nucleoside. In some embodiments the modification of the internucleotide linkage comprises replacement of one or more oxygens of the phosphodiester linkage. In some embodiments the modification of the internucleotide linkage comprises a modification at one or more oxygens of the phosphodiester linkage.

In some embodiments, the sugar is modified by O-alkylation (e.g., O-methylation), by the replacement or substitution of an atom of the sugar with a group selected from optionally substituted amino, optionally substituted thiol, optionally substituted azido, optionally substituted alkyl, optionally substituted alkenyl, and halo, or the replacement of an —O— with an —S—. In some embodiments, the chemical modification to the sugar-phosphate backbone replaces one or more oxygens of the phosphodiester linkage with a group selected from amino, S, and BH₃.

In some embodiments of the present disclosure where the donor moiety is an RNA, the RNA is an mRNA.

In some embodiments of the present disclosure, the 5′-end of the donor moiety comprises a 5′cap. In some embodiments, the 5′-end of the donor moiety is a 5′-untranslated region (UTR). In some embodiments where the donor moiety is an mRNA, the mRNA has a 5′ cap. In some embodiments, the mRNA has a 5′ UTR.

In some embodiments of the present disclosure, the leaving group is a 5′-monophosphate group. In some embodiments, the leaving group is a 5′-AppN group.

In some embodiments, the donor comprises a modified 3′-end. In some embodiments, the modified 3′-end comprises a modification that enhances purification. In some embodiments, the modified 3′-end comprises a modification to enhance resistance to nucleases. In some embodiments, the modified 3′-end comprises a modification to enhance ease of visualization. In some embodiments, the modified 3′-end comprises a detectable agent. In some embodiments, the modified 3′-end comprises a fluorophore.

In some embodiments of the present disclosure, the acceptor moiety comprises one nucleotide. In some embodiments, the acceptor moiety comprises more than one nucleotide. In some embodiments, the acceptor moiety is between about two and about 850 nucleotides in length. In some embodiments, the acceptor moiety is more than 850 nucleotides in length.

In some embodiments of the present disclosure, the acceptor moiety is a dinucleotide. In some embodiments, the acceptor moiety comprises a trinucleotide. In some embodiments, the acceptor moiety comprises an mRNA cap. In some embodiments, the acceptor moiety comprises a cap-like structure. In some embodiments, the acceptor moiety comprises a non-cap like structure.

In some embodiments of the present disclosure wherein the acceptor moiety is a dinucleotide, the dinucleotide contains a 5′-triphosphate group. In some embodiments wherein the acceptor moiety is a dinucleotide, the dinucleotide contains a 5′-inverted guanosine group.

In some embodiments, the donor moiety comprises between one and about 10000 nucleotides. In some embodiments, the donor moiety comprises more than 10000 nucleotides.

In some embodiments of the present disclosure, the donor moiety is a chemically synthesized RNA. In some embodiments, the donor moiety is an enzymatically synthesized RNA.

In some embodiments, the methods of the disclosure further comprise purifying the modified RNA. In some embodiments, the purification resolves the modified RNA from unreacted donor moiety. In some embodiments, the purification comprises enzymatically degrading the unreacted donor moiety. In some embodiments, the unreacted donor moiety is enzymatically degraded using an exonuclease specific for 5′-monophosphate-containing RNA. In some embodiments, this exonuclease is exonuclease is XRN-1.

In some embodiments, the purification of the modified RNA separates the modified RNA from the unreacted acceptor moiety. In some embodiments, separation of the modified RNA from the unreacted acceptor moiety is carried out using ultra-filtration. In some embodiments, separation of the modified RNA from the unreacted acceptor moiety is carried out with chromatographic methods.

In some embodiments, the purification of the modified RNA comprises the use of an affinity tag. In some embodiments, the affinity tag is a chemical tag. In some embodiments, the affinity tag is an oligonucleotide tag. In some embodiments, the affinity tag is a 5′ cap.

In some embodiments wherein the purification comprises an oligonucleotide tag, the sequence of the oligonucleotide is a poly(A) sequence. In some embodiments, the sequence of the oligonucleotide is an MS2 protein binding sequence. In some embodiments, the sequence of the oligonucleotide is an aptamer sequence. In some embodiments the aptamer binds to Streptavidin. In some embodiments the aptamer binds to Sephadex. In some embodiments, purification of an RNA of the disclosure (e.g., a modified RNA of the disclosure, or a modified RNA made by the methods of the disclosure) comprises the use of a 5′ cap. In some embodiment, the 5′ cap is a 5′ 7-methyl guanosine cap.

In some embodiments of the disclosure, a modified RNA prepared by a method of the disclosure is a modified mRNA.

In some embodiments, the enzymatic ligation further comprises the use of a single stranded DNA (ssDNA) splint. In some embodiments of the present disclosure, the ssDNA splint comprises a sequence complementary to at least one base pair at the 3′-end of the acceptor moiety or at least one basepair at the 5′-end of the donor moiety. In some embodiments, the ssDNA splint comprises a sequence complementary to at least one base pair at the 3′-end of the acceptor moiety and at least one basepair at the 5′-end of the donor moiety. In some embodiments, the ssDNA splint comprises a DNA sequence complementary to at least 1, 2, 3, 4, 5, 6, or 7 basepairs at the 3′-end of the acceptor moiety. In some embodiments, the ssDNA splint comprises a DNA sequence complementary to at least 1, 2, 3, 4, 5, 6, or 7 basepairs at the 5′ end of the donor moiety. In some embodiments, the ssDNA splint comprises a DNA sequence complementary to between 1 and 20 basepairs at the 3′ end of the acceptor moiety. In some embodiments, the ssDNA splint comprises a DNA sequence complementary to between 1 and 20 basepairs at the 5′ end of the donor moiety. In some embodiments, the ssDNA splint comprises a DNA sequence complementary to between 21 and 40 basepairs at the 3′ end of the acceptor moiety. In some embodiments, the ssDNA splint comprises a DNA sequence complementary to between 21 and 40 basepairs at the 5′ end of the donor moiety. In some embodiments, the ssDNA splint comprises a DNA sequence complementary to between 41 and 60 basepairs at the 3′ end of the acceptor moiety. In some embodiments, the ssDNA splint comprises a DNA sequence complementary to between 41 and 60 basepairs at the 5′ end of the donor moiety. In some embodiments, the ssDNA splint comprises a DNA sequence complementary to at least 20 basepairs at the 3′ end of the acceptor moiety. In some embodiments, the ssDNA splint comprises a DNA sequence complementary to between at least 20 basepairs at the 5′ end of the donor moiety.

In some embodiments, the ssDNA splint comprises a DNA sequence complementary to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs at the 3′ end of the acceptor moiety. In some embodiments, the ssDNA splint comprises a DNA sequence complementary to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs at the 3′ end of the donor moiety.

In some embodiments, the ssDNA splint is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 basepairs long.

In some embodiments, the length of the DNA sequence complementary to the 3′ end of the acceptor moiety and the length of the sequence complementary to the 5′ end of the donor moiety are not the same. In some embodiments, the length of the DNA sequence complementary to the 3′-end of the acceptor moiety and the length of the sequence complementary to the 5′-end of the donor moiety are the same. In some embodiments, the sequence of complementary to the 3′-end of the acceptor moiety is offset from the 3′ terminus of the acceptor moiety by at least one basepair. In some embodiments, the acceptor moiety comprises a sequence at least 2 nucleotides in length. In some embodiments, there is at least one mismatch between the sequence of the ssDNA splint and the 3′-end of the acceptor moiety. In some embodiments, the 3′ end of the acceptor moiety and the 5′-end of the donor moiety form an RNA stem-loop.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

The patent or application file contains at least one drawing executed in color. Copies of this patent or patent application publication with color drawing(s) will be provided by the Office upon request and payment of the necessary fee.

The above and further features will be more clearly appreciated from the following detailed description when taken in conjunction with the accompanying drawings.

FIG. 1A is a general scheme illustrating RNA ligation for the installation of an acceptor moiety, showing a non-splinted RNA to RNA ligation. The ligation reaction is enzymatically catalyzed by T4 RNA Ligase 1. The Ligation donor has a 5′-monophosphate group and the ligation acceptor has a 3′-hydroxyl group;

FIG. 1B illustrates RNA ligation for the installation of an acceptor moiety in RNA, and shows the basic mechanism of RNA to RNA ligation catalyzed by T4 RNA Ligase 1. The ligation results in a natural phosphodiester backbone linkage bridging the RNA at the site of ligation.

FIG. 1C illustrates the installation of an acceptor moiety in RNA. R₁ can be a hydrogen or a cap-like structure such as a guanosine or analogue linked to the triphosphate (in blue) at the 5′-carbon of the guanosine, or another chemical entity. R₂, R₃, and/or R₄ can be a hydrogen or other chemical moieties such as a methyl group of a fluorine.

FIG. 1D is a general scheme illustrating RNA ligation for the installation of an acceptor moiety in RNA, summarizing the recovery of pure RNA product from the installation of an acceptor moiety in RNA. Treatment with XRN-1 removes remaining unligated donor and subsequent ultrafiltration removes remaining unligated acceptor (denoted in the scheme by ‘A’).

FIG. 2 is a schematic illustrating the reaction design of the exemplary ligation reaction described in Example 2.

FIG. 3 is a graph showing percentages of activated B-cells in the spleens of black mice dosed with mRNAs produced by the ligation reaction described in Example 2. PBS is used as control. The Figure suggests that, as long as the input material is purified by reverse phase chromatography, the immune response appears to be no different than the control response. Numbers 1-10 refer the following: 1: ligation product purified by dT; input material purified by dT; 2: ligation product purified by reverse phase chromatography; input material purified by dT; 3: ligation product purified by dT; input material purified by reverse phase chromatography; 4: ligation product purified by reverse phase chromatography; input material purified by reverse phase chromatography; 5: ligation product purified by reverse phase chromatography using tris(hydroxymethyl)aminomethane; input material purified by reverse phase chromatography; 6: dT control; 7: reverse phase chromatography control; 8: process control; 9: PBS; 10: treatment naïve subject.

FIG. 4 is a graph showing expression of huEpo in black mice 6 h and 24 h after being dosed with mRNAs produced by the ligation reaction described in Example 2. PBS is used as control. The Figure suggests that, as long as the input material is purified by reverse phase chromatography, the immune response appears to be no different than the control response. Numbers 1-9 refer the following: 1: ligation product purified by dT; input material purified by dT; 2: ligation product purified by reverse phase chromatography; input material purified by dT; 3: ligation product purified by dT; 4: ligation product purified by reverse phase chromatography; input material purified by reverse phase chromatography; 5: ligation product purified by reverse phase chromatography using tris(hydroxymethyl)aminomethane, input material purified by reverse phase chromatography; 6: dT control; 7: reverse phase chromatography control; 8: process control; 9: PBS.

FIG. 5A is a schematic illustrating modification types and positions of modifications in an acceptor moiety (SEQ ID No. 4), used in a ligation conducted to demonstrate installation of a modified 5′-untranslated region to an mRNA donor.

FIG. 5B is a graph showing expression in HEK293 cells of various mRNA ligation products comprising 5′-untranslated regions with modifications as illustrated in FIG. 5A. Full length mRNAs that were exposed to the exact same process as the ligated samples were used as a control to ensure that the process was not itself damaging mRNA. Expression of the process controls (identical duplicates v1A and v1B) was comparable to expression of material made by in vitro transcription (IVT). Furthermore, several of the materials comprising modifications (e.g. L3-L5 in the Figure), expressed on par with the unmodified ligated mRNA (L1).

FIG. 6 is a series of schemes illustrating alternatives to splinted ligations. In the stem loop ligation alternative, the polynucleotide on the left (SEQ ID NO: 5) is ligated to the Cy3-labeled polynucleotide on the right within the loop region to produce a single polynucleotide (SEQ ID NO: 6).

FIG. 7A is a schematic illustrating overlap ratios for splints for 5′-ligations with T4 RNA Ligase 1 (RNL1).

FIG. 7B is a schematic illustration of the experimental setup for testing splints that promote a variety of symmetrical and asymmetrical overlaps.

FIG. 7C is an image showing the results of a splint screen assay using fluoroprobes (Bio-Rad Chemidoc) testing splints as described in FIGS. 7A and 7B. Reactions were terminated before completion. 6% PAGE-D ran at 180V for 24 min. All samples were DNase treated before being annealed to Left Probe at 65° C. for 5 min.

FIG. 8 is a schematic illustration of a construct design for stem-loop directed ligations with T4 RNA Ligase 1. The ligase was tested for its ability to promote ligations by engineering a stem-loop into a untranslated region.

FIG. 9 is an image showing the results of an assay using fluoroprobes containing 2′-O methylated DNA oligonucleotides designed to hybridize the full length mRNA product, to screen 3′ end ligations with splints and stem-loops. The reactions ran in triplicate for 1 hour with T4 RNA Ligase 1 and no PEG. The results show that the stem-loop system affords quantitative conversion, while some yield was observed with overhang splint as well.

FIG. 10 is a graph showing that mass spectrometry reveals that T7RNP resulted in untemplated additions onto the 3′-end of RNA transcripts. The nucleotide added does not appear to depend on the identity of its nucleobase.

FIG. 11 is a series of illustrations showing examples of dsDNA templates investigated for the purpose of reducing untemplated nucleotide addition at the 3′-end of the resulting transcript.

FIG. 12A is a scheme illustrating the ligation of rightmer and leftmer RNA on a DNA splint, showing that even a single nucleotide overhang at the ligation site predictably and entirely abolishes ligation efficiently.

FIG. 12B is a scheme illustrating the ligation of rightmer and leftmer RNA on a DNA splint.

FIG. 13 is a graph showing a PAGE-D gel illustrating ligation efficiency for rightmers produced with different polymerases. Ligation yield was calculated as a ratio of bands corresponding to unligated rightmer and ligated full-length product. The reaction was terminated early to exaggerate differences in reaction yields.

FIG. 14 is a graph showing expression of huEpo in black mice 6 h and 24 h after being dosed with mRNAs produced by the ligation reaction described in Example 2. The Figure suggests that affinity chromatography is a superior purification method to enzymatic purification in terms of post-purification expression levels. Numbers 1-8 refer the following: 1: non-ligated control, purified by reverse phase chromatography; 2: Process control 1; 3: Process control 2; 4: ligation product purified by DNAse, Xrn-1, and dT; 5: ligation product purified DNAse, Xrn-1, and chromatography using tris(hydroxymethyl)aminomethane; 6 ligation product purified by DNAse, RppH, Xrn-1, and dT; 7: ligation product purified by DNAse and 5′ affinity chromatography; 8: ligation product purified by DNAse, dT, and 5′ affinity chromatography

FIG. 15 is a graph showing percentages of activated B-cells in the spleens of black mice dosed with mRNAs produced by the ligation reaction described in Example 2. PBS is used as control. The Figure suggests that, in every iteration of purification, the immune response appears to be no different than the control response. N refers to naïve mice; P refers to mice injected only with PBS; Numbers 1-8 refer the following: 1: non-ligated control, purified by reverse phase chromatography; 2: Process control 1; 3: Process control 2; 4: ligation product purified by DNAse, Xrn-1, and dT; 5: ligation product purified DNAse, Xrn-1, and chromatography using tris(hydroxymethyl)aminomethane; 6 ligation product purified by DNAse, RppH, Xrn-1, and dT; 7: ligation product purified by DNAse and 5′ affinity chromatography; 8: ligation product purified by DNAse, dT, and 5′ affinity chromatography

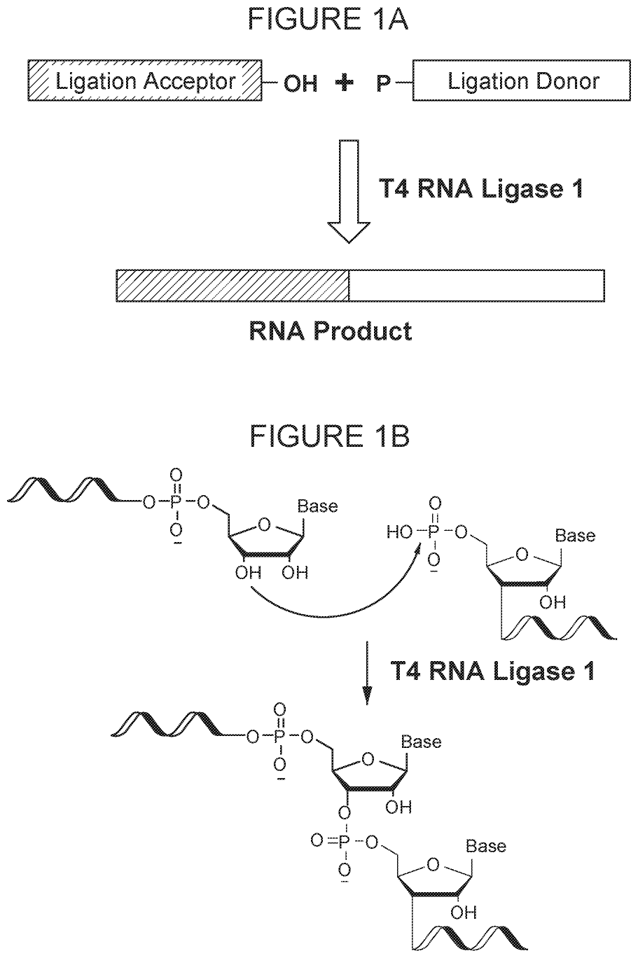

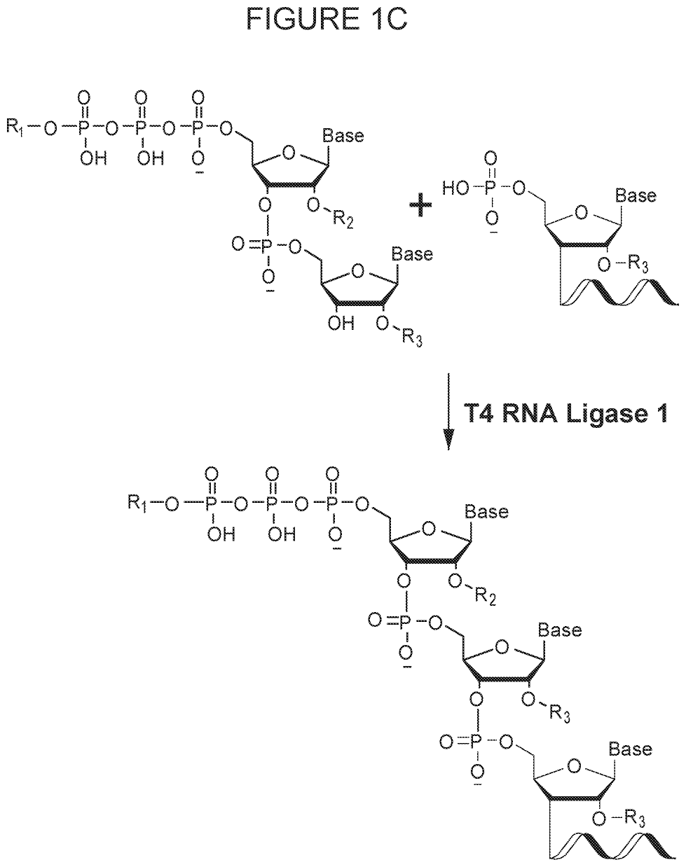

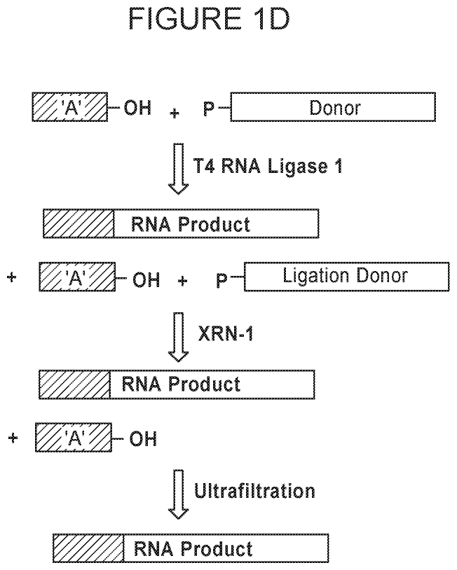

### DETAILED DESCRIPTION

The present disclosure relates to methods of preparing a modified RNA, including, for example installation of 5′-triphosphate groups, mRNA caps (including modified mRNA caps), cap like structures or non-cap like structures on RNA.

In some aspects, the present disclosure provides a method of preparing a modified RNA by enzymatically ligating an acceptor moiety to a donor moiety. In some embodiments, the present disclosure provides a method of preparing a modified RNA by enzymatically ligating an acceptor moiety to the 5′-end of a donor moiety. In some embodiments, the present disclosure provides a method of preparing a modified RNA by enzymatically ligating the 3′-end of an acceptor moiety to the 5′-end of a donor moiety. In some embodiments, the acceptor moiety has a 3′-hydroxyl group. In some embodiments, the donor moiety has a leaving group.

In further aspects, the present disclosure provides a modified RNA prepared by the methods disclosed herein. In some embodiments, the modified RNA is prepared by enzymatically ligating an acceptor moiety to a donor moiety. In some embodiments, the modified RNA is prepared by enzymatically ligating an acceptor moiety to the 5′-end of a donor moiety. In some embodiments, the modified RNA is prepared by enzymatically ligating the 3′-end of an acceptor moiety to the 5′-end of a donor moiety. In some embodiments, the acceptor moiety has a 3′-hydroxyl group. In some embodiments, the donor moiety has a leaving group.

In some embodiments, this enzymatic ligation is performed in a single enzymatic step.

In some embodiments, the single enzymatic step comprises the use of a ligation enzyme. In some embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, and combinations thereof.

In some embodiments, the ligation enzyme is T4 DNA ligase. In some embodiments, the ligation enzyme is T4 RNA ligase 1. In some embodiments, the ligation enzyme is T4 RNA ligase 2. In some embodiments, the T4 RNA ligase 2 is truncated. In some embodiments, the T4 RNA ligase 2 is truncated. In some embodiments, the T4 RNA ligase 2 has a K227Q mutation. In some embodiments, the T4 RNA ligase 2 has a R55K mutation. In some embodiments, the T4 RNA ligase 2 is truncated and has a K227Q mutation. In some embodiments, the T4 RNA ligase 2 is truncated and has a R55K mutation. In some embodiments, the T4 RNA ligase 2 is truncated and has both a R55K mutation and a K227Q mutation. In some embodiments, the ligation enzyme is RtcB ligase. In some embodiments, the ligation enzyme is T3 DNA ligase. In some embodiments, the ligation enzyme is T7 DNA ligase. In some embodiments, the ligation enzyme is Taq DNA ligase. In some embodiments, the ligation enzyme is PBCV-1 DNA Ligase (i.e, Chlorella virus DNA Ligase; SplintR® ligase). In some embodiments, the ligation enzyme is a thermostable DNA ligase. In some embodiments, the thermostable ligase is 5′AppDNA/RNA ligase. In some embodiments, the ligation enzyme is an ATP dependent DNA ligase. In some embodiments, the ATP dependent DNA ligase is 9° N® DNA ligase. In some embodiments, the enzymatic ligation is performed by a mixture of ligases.

In some embodiments of the present disclosure, the donor moiety is an RNA. In some embodiments, the donor moiety is an RNA comprising a non-naturally occurring nucleotide. In some embodiments of the present disclosure, a non-naturally occurring nucleotide comprises one or more chemical modifications of a naturally occurring nucleotide.

In some embodiments of the present disclosure, the naturally occurring nucleotide comprises chemical modifications that are located on the major groove face of the nucleobase portion of the nucleotide. In some embodiments, the chemical modification replaces an atom of the major groove face of the nucleobase with a group selected from optionally substituted amino, optionally substituted thiol, optionally substituted alkyl, optionally substituted alkenyl, and halo. In some embodiments, the chemical modification replaces an atom of the major groove face of the nucleobase with a substituted or unsubstituted amino. In some embodiments, the chemical modification replaces an atom of the major groove face of the nucleobase with a substituted or unsubstituted thiol. In some embodiments, the chemical modification replaces an atom of the major groove face of the nucleobase with a substituted or unsubstituted alkyl. In some embodiments, the chemical modification replaces an atom of the major groove face of the nucleobase with substituted or unsubstituted alkenyl. In some embodiments, the chemical modification replaces an atom of the major groove face of the nucleobase with halo. In some embodiments, the nucleobase portion of the non-naturally occurring nucleotide is a pyrimidine nucleobase. For example, in some embodiments, the non-naturally occurring nucleotide is selected from the group consisting of cytosine (C), thymine (T), and uracil (U).

In some embodiments of the present disclosure, the non-naturally occurring nucleotide comprises chemical modifications located on the sugar. In some embodiments, a modification is a modification at the 2′ position of the nucleoside. In some embodiments, the chemical modification is 2′-O alkylation. In some embodiments, the chemical modification at the 2′ position is 2′-O methylation. In some embodiments, the chemical modification replaces an atom of the sugar with a group selected from optionally substituted amino, optionally substituted thiol, optionally substituted azido, optionally substituted alkyl, optionally substituted alkenyl, and halo. In some embodiments, the chemical modification replaces an atom of the sugar with a substituted or unsubstituted amino. In some embodiments, the chemical modification replaces an atom of the sugar with a substituted or unsubstituted thiol. In some embodiments, the chemical modification replaces an atom of the sugar with a substituted or unsubstituted azido. In some embodiments, the chemical modification replaces an atom of the sugar with a substituted or unsubstituted alkyl. In some embodiments, the chemical modification replaces an atom of the sugar with a substituted or unsubstituted alkenyl. In some embodiments, the chemical modification replaces an atom of the sugar with halo.

In some embodiments of the present disclosure, the modification located on the sugar comprises a modification at the 4′ position of the nucleoside. In some embodiments, the chemical modification replaces an atom of the sugar with a group selected from optionally substituted amino, optionally substituted thiol, optionally substituted azido, optionally substituted alkyl, optionally substituted alkenyl, and halo. In some embodiments, the chemical modification replaces an atom of the sugar with a substituted or unsubstituted amino. In some embodiments, the chemical modification replaces an atom of the sugar with a substituted or unsubstituted thiol. In some embodiments, the chemical modification replaces an atom of the sugar with a substituted or unsubstituted azido. In some embodiments, the chemical modification replaces an atom of the sugar with a substituted or unsubstituted alkyl. In some embodiments, the chemical modification replaces an atom of the sugar with a substituted or unsubstituted alkenyl. In some embodiments, the chemical modification replaces an atom of the sugar with halo.

In some embodiments of the present disclosure, the sugar is modified at both the 2′ and 4′ positions of the nucleoside. In some embodiments, the chemical modification at the 2′ position is 2′-O alkylation. In some embodiments, the chemical modification at the 2′-O methylation. In some embodiments, the chemical modification at the 2′ or 4′ position replaces an atom of the sugar with a group selected from optionally substituted amino, optionally substituted thiol, optionally substituted azido, optionally substituted alkyl, optionally substituted alkenyl, and halo. In some embodiments, the chemical modification replaces an atom of the sugar with a substituted or unsubstituted amino. In some embodiments, the chemical modification replaces an atom of the sugar with a substituted or unsubstituted thiol. In some embodiments, the chemical modification replaces an atom of the sugar with a substituted or unsubstituted azido. In some embodiments, the chemical modification replaces an atom of the sugar with a substituted or unsubstituted alkyl. In some embodiments, the chemical modification replaces an atom of the sugar with a substituted or unsubstituted alkenyl. In some embodiments, the chemical modification replaces an atom of the sugar with halo.

In some embodiments of the present disclosure, the donor moiety is an RNA with one or more chemical modifications located on the sugar-phosphate backbone. In some embodiments, the chemical modification on the sugar-phosphate backbone comprises replacing one or more oxygens of the phosphodiester linkage with an amine, S, or BH₃. In some embodiments one or more oxygens of the phosphodiester linkage are replaced with a group selected from S and BH₃.

In some embodiments of the present disclosure, the donor RNA comprises one or more chemical modifications of the sugar and the internucleotide linkage of the donor RNA. In some embodiments, donor RNA comprises a modification of the sugar at the 2′-position of the nucleoside. In some embodiments, donor RNA comprises a modification of the sugar at the 4′-position of the nucleoside. In some embodiments, donor RNA comprises a modification of the sugar at the 2′-position and the 4′-position of the nucleoside. In some embodiments the modification of the internucleotide linkage comprises replacement of one or more oxygens of the phosphodiester linkage. In some embodiments the modification of the internucleotide linkage comprises substitution of one or more oxygens of the phosphodiester linkage. In some embodiments, the sugar is modified by O-alkylation (e.g., O-methylation), by the replacement or substitution of an atom of the sugar with a group selected from optionally substituted amino, optionally substituted thiol, optionally substituted azido, optionally substituted alkyl, optionally substituted alkenyl, and halo, or the replacement of an —O— with an —S—. In some embodiments, the chemical modification to the sugar-phosphate backbone replaces one or more oxygens of the phosphodiester linkage with an amine, S, or BH₃.

In some embodiments of the present disclosure, a substituted amino is selected from the group consisting of alkylamino, dialkylamino, alkenylamino, dialkenylamino, alkylalkenylamino, arylamino, diarylamino, alkylarylamino and arylalkenylamino. In some embodiments, the alkylamino, dialkylamino, alkenylamino, dialkenylamino, alkylalkenylamino, arylamino, diarylamino, alkylarylamino or arylalkenylamino are further substituted.

In some embodiments of the present disclosure, a substituted thiol is selected from the group consisting of alkylthio, alkenylthio, sulfonyl, alkylsulfonyl, sulfinyl, alkylsulfinyl and sulfamoyl. In some embodiments, an alkylthio, alkenylthio, sulfonyl, alkylsulfonyl, sulfinyl, alkylsulfinyl or sulfamoyl is further substituted.

In some embodiments of the present disclosure, a substituted azido is selected from the group consisting of alkylazido, dialkylazido, alkenylazido, dialkenylazido, alkylalkenylazido, arylazido, diarylazido, alkylarylazido and arylalkenylazido. In some embodiments, alkylazido, dialkylazido, alkenylazido, dialkenylazido, alkylalkenylazido, arylazido, diarylazido, alkylarylazido or arylalkenylazido are further substituted.

In some embodiments of the present disclosure, an alkyl is selected from the group consisting of methyl, ethyl, i-propyl, and n-propyl.

In some embodiments of the present disclosure, an alkenyl is selected from the group consisting of ethenyl, i-propenyl, and n-propenyl.

In some embodiments of the present disclosure, a halo is selected from the group consisting of chloro, fluoro, bromo, and iodo.

In some embodiments of the present disclosure, the donor moiety is an RNA. In some embodiments, the donor moiety is an mRNA.

In some embodiments of the present disclosure, the 5′-end of the donor moiety has a 5′cap. In some embodiments, the 5′-end of the donor moiety is a 5′-untranslated region. In some embodiments, where the donor moiety is an mRNA, the mRNA has a 5′ cap. In some embodiments, the mRNA has a 5′ UTR.

In some embodiments of the present disclosure, the leaving group is a 5′-monophosphate group. In some embodiments, the leaving group is a 5′-AppN group.

In some embodiments, the donor comprises a modified 3′-end. In some embodiments, the modified 3′-end comprises a modification that enhances purification. In some embodiments, the modified 3′-end comprises a modification to enhance resistance to nucleases. In some embodiments, the modified 3′-end comprises a modification to enhance ease of visualization. In some embodiments, the modified 3′-end comprises a detectable agent. In some embodiments, the modified 3′-end comprises a fluorophore. For example, in some embodiments, the fluorophore is selected from the group consisting of Cy3, Cy3.5, Cy5, Cy5.5, Cy7, GFP, and IR783.

In some embodiments of the present disclosure, the acceptor moiety comprises one nucleotide. In some embodiments, the acceptor moiety comprises more than one nucleotide. In some embodiments, the acceptor moiety is between about two and about 850 nucleotides in length. In some embodiments, the acceptor moiety is between two and 10 nucleotides in length. In some embodiments, the acceptor moiety is between 11 and 30 nucleotides in length. In some embodiments, the acceptor moiety is between 30 and 40 nucleotides in length. In some embodiments, the acceptor moiety is between 41 and 50 nucleotides in length. In some embodiments, the acceptor moiety is between 51 and 60 nucleotides in length. In some embodiments, the acceptor moiety is between 61 and 80 nucleotides in length. In some embodiments, the acceptor moiety is between 81 and 100 nucleotides in length. In some embodiments, the acceptor moiety is between 101 and 120 nucleotides in length. In some embodiments, the acceptor moiety is between 121 and 140 nucleotides in length. In some embodiments, the acceptor moiety is between 141 and 160 nucleotides in length. In some embodiments, the acceptor moiety is between 161 and 180 nucleotides in length. In some embodiments, the acceptor moiety is between 181 and 200 nucleotides in length. In some embodiments, the acceptor moiety is between 201 and 250 nucleotides in length. In some embodiments, the acceptor moiety is between 251 and 300 nucleotides in length. In some embodiments, the acceptor moiety is between 301 and 350 nucleotides in length. In some embodiments, the acceptor moiety is between 401 and 450 nucleotides in length. In some embodiments, the acceptor moiety is between 451 and 500 nucleotides in length. In some embodiments, the acceptor moiety is between 501 and 550 nucleotides in length. In some embodiments, the acceptor moiety is between 551 and 600 nucleotides in length. In some embodiments, the acceptor moiety is between 601 and 650 nucleotides in length. In some embodiments, the acceptor moiety is between 651 and 700 nucleotides in length. In some embodiments, the acceptor moiety is between 701 and 750 nucleotides in length. In some embodiments, the acceptor moiety is between 751 and 800 nucleotides in length. In some embodiments, the acceptor moiety is between 801 and 850 nucleotides in length. In some embodiments, the acceptor moiety is more than 850 nucleotides in length.

In some embodiments of the present disclosure, the acceptor moiety is a dinucleotide. In some embodiments, the acceptor moiety comprises a trinucleotide. In some embodiments, the acceptor moiety comprises an mRNA cap. In some embodiments, the acceptor moiety comprises a cap-like structure. In some embodiments, the acceptor moiety comprises a non-cap like structure.

In some embodiments of the present disclosure, wherein the acceptor moiety is a dinucleotide, the dinucleotide contains a 5′-triphosphate group. In some embodiments, where the acceptor moiety is a dinucleotide, the dinucleotide contains a 5′-inverted guanosine group.

In some embodiments, the donor moiety comprises between one and about 10000 nucleotides. In some embodiments, the donor moiety is between one and 10 nucleotides in length. In some embodiments, the donor moiety is between 11 and 30 nucleotides in length. In some embodiments, the donor moiety is between 30 and 40 nucleotides in length. In some embodiments, the donor moiety is between 41 and 50 nucleotides in length. In some embodiments, the donor moiety is between 51 and 60 nucleotides in length. In some embodiments, the donor moiety is between 61 and 80 nucleotides in length. In some embodiments, the donor moiety is between 81 and 100 nucleotides in length. In some embodiments, the donor moiety is between 101 and 120 nucleotides in length. In some embodiments, the donor moiety is between 121 and 140 nucleotides in length. In some embodiments, the donor moiety is between 141 and 160 nucleotides in length. In some embodiments, the donor moiety is between 161 and 180 nucleotides in length. In some embodiments, the donor moiety is between 181 and 200 nucleotides in length. In some embodiments, the donor moiety is between 201 and 250 nucleotides in length. In some embodiments, the donor moiety is between 251 and 300 nucleotides in length. In some embodiments, the donor moiety is between 301 and 350 nucleotides in length. In some embodiments, the donor moiety is between 401 and 450 nucleotides in length. In some embodiments, the donor moiety is between 451 and 500 nucleotides in length. In some embodiments, the donor moiety is between 501 and 550 nucleotides in length. In some embodiments, the donor moiety is between 551 and 600 nucleotides in length. In some embodiments, the donor moiety is between 601 and 650 nucleotides in length. In some embodiments, the donor moiety is between 651 and 700 nucleotides in length. In some embodiments, the donor moiety is between 701 and 750 nucleotides in length. In some embodiments, the donor moiety is between 751 and 800 nucleotides in length. In some embodiments, the donor moiety is between 801 and 850 nucleotides in length. In some embodiments, the donor moiety is between 851 and 900 nucleotides in length. In some embodiments, the donor moiety is between 901 and 950 nucleotides in length. In some embodiments, the donor moiety is between 951 and 1000 nucleotides in length. In some embodiments, the donor moiety is between 1001 and 2000 nucleotides in length. In some embodiments, the donor moiety is between 2001 and 3000 nucleotides in length. In some embodiments, the donor moiety is between 3001 and 4000 nucleotides in length. In some embodiments, the donor moiety is between 4001 and 5000 nucleotides in length. In some embodiments, the donor moiety is between 5001 and 6000 nucleotides in length. In some embodiments, the donor moiety is between 6001 and 7000 nucleotides in length. In some embodiments, the donor moiety is between 7001 and 8000 nucleotides in length. In some embodiments, the donor moiety is between 8001 and 9000 nucleotides in length. In some embodiments, the donor moiety is between 9001 and 10000 nucleotides in length. In some embodiments, the donor moiety is more than 10000 nucleotides in length.

In some embodiments of the present disclosure, the donor moiety is a chemically synthesized RNA. In some embodiments, the donor moiety is an enzymatically synthesized RNA.

In some embodiments, the methods of the disclosure further comprise purifying the modified RNA. In some embodiments, the purification resolves the modified RNA from unreacted donor moiety. In some embodiments, the purification comprises enzymatically degrading the unreacted donor moiety. In some embodiments, the unreacted donor moiety is enzymatically degraded using an exonuclease specific for 5′-monophosphate-containing RNA. In some embodiments, this exonuclease is exonuclease is XRN-1.

In some embodiments, the purification of the modified RNA separates the modified RNA from the unreacted acceptor moiety. In some embodiments, separation of the modified RNA from the unreacted acceptor moiety is carried out using ultra-filtration. In some embodiments, separation of the modified RNA from the unreacted acceptor moiety is carried out with chromatographic methods. In some embodiments, chromatographic methods of the disclosure comprise, e.g., strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

In some embodiments purification of an RNA of the disclosure comprises the use of an affinity tag. In some embodiments, the affinity tag is a chemical tag. In some embodiments, the chemical tag comprises biotin. In some embodiments, the affinity tag is an oligonucleotide tag. In some embodiments, the affinity tag is a 5′ cap.

In some embodiments of the disclosure wherein the affinity tag is an oligonucleotide tag, the sequence of the oligonucleotide is a poly(A) sequence. In some embodiments, the sequence of the oligonucleotide is an MS2 protein binding sequence. In some embodiments, the sequence of the oligonucleotide is an aptamer sequence. In some embodiments the aptamer binds to Streptavidin. In some embodiments the aptamer binds to Sephadex. In some embodiments, purification of an RNA of the disclosure (e.g., a modified RNA of the disclosure, or a modified RNA made by the methods of the disclosure) comprises the use of a 5′ cap. In some embodiment, the 5′ cap is a 5′ 7-methyl guanosine cap.

In some embodiments of the disclosure, the modified RNA prepared by enzymatically ligating an acceptor moiety to the 5′-end of a donor moiety and purification is a modified mRNA. In some embodiments, the modified RNA prepared by enzymatically ligating an acceptor moiety to the 5′-end of a donor moiety and purification is a modified RNA.

In some embodiments, the enzymatic ligation further comprises the use of a single stranded DNA (ssDNA) splint. In some embodiments of the present disclosure, the ssDNA splint comprises a sequence complementary to at least one base pair at the 3′-end of the acceptor moiety or at least one basepair at the 5′-end of the donor moiety. In some embodiments, the ssDNA splint comprises a sequence complementary to at least one base pair at the 3′-end of the acceptor moiety and at least one basepair at the 5′-end of the donor moiety. In some embodiments, the ssDNA splint comprises a DNA sequence complementary to at least 1, 2, 3, 4, 5, 6, or 7 basepairs at the 3′-end of the acceptor moiety. In some embodiments, the ssDNA splint comprises a DNA sequence complementary to at least 1, 2, 3, 4, 5, 6, or 7 basepairs at the 5′ end of the donor moiety. In some embodiments, the ssDNA splint comprises a DNA sequence complementary to between 1 and 20 basepairs at the 3′ end of the acceptor moiety. In some embodiments, the ssDNA splint comprises a DNA sequence complementary to between 1 and 20 basepairs at the 5′ end of the donor moiety. In some embodiments, the ssDNA splint comprises a DNA sequence complementary to between 21 and 40 basepairs at the 3′ end of the acceptor moiety. In some embodiments, the ssDNA splint comprises a DNA sequence complementary to between 21 and 40 basepairs at the 5′ end of the donor moiety. In some embodiments, the ssDNA splint comprises a DNA sequence complementary to between 41 and 60 basepairs at the 3′ end of the acceptor moiety. In some embodiments, the ssDNA splint comprises a DNA sequence complementary to between 41 and 60 basepairs at the 5′ end of the donor moiety. In some embodiments, the ssDNA splint comprises a DNA sequence complementary to at least 20 basepairs at the 3′ end of the acceptor moiety. In some embodiments, the ssDNA splint comprises a DNA sequence complementary to between at least 20 basepairs at the 5′ end of the donor moiety.

In some embodiments, the ssDNA splint comprises a DNA sequence complementary to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs at the 3′ end of the acceptor moiety. In some embodiments, the ssDNA splint comprises a DNA sequence complementary to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs at the 3′ end of the donor moiety.

In some embodiments, the ssDNA splint is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 basepairs long.

In some embodiments, the length of the DNA sequence complementary to the 3′ end of the acceptor moiety and the length of the sequence complementary to the 5′ end of the donor moiety are not the same. In some embodiments, the length of the DNA sequence complementary to the 3′-end of the acceptor moiety and the length of the sequence complementary to the 5′-end of the donor moiety are the same. In some embodiments, the sequence of complementary to the 3′-end of the acceptor moiety is offset from the 3′ terminus of the acceptor moiety by at least one basepair. In some embodiments, the acceptor moiety comprises a sequence at least 2 nucleotides in length. In some embodiments, there is at least one mismatch between the sequence of the ssDNA splint and the 3′-end of the acceptor moiety. In some embodiments, the 3′ end of the acceptor moiety and the 5′-end of the donor moiety form an RNA stem-loop.

### Purification Methods

In some embodiments, ultra-filtration refers to a membrane separation technique used to separate extremely small particles and molecules in fluids. In ultra-filtration, molecules or particles are primarily separated based on size, although other factors such as the shape and charge of the molecules also play a role. Molecules larger than the pores of the membrane will be retained on the membrane, while molecules smaller than the pores of the membrane will pass through, separating the two sizes of molecules. Ultra-filtration is most effective when the molecules being separated differ in size by at least an order of magnitude.

In some embodiments, chromatographic methods refers to passing the mixture of molecules in disclosed in the invention through a medium in which the molecules move at different rates. In some embodiments, RNAs are purified using either size exclusion chromatography or affinity chromatography. In some embodiments, size exclusion chromatography uses a high-resolution fast performance liquid chromatography system (FPLC) to remove small unincorporated ribonucleotides and other small transcripts. In some embodiments, affinity chromatographic methods use an affinity tag to separate out the desired molecules.

In some embodiments the affinity tag is a chemical tag. One such chemical tag is biotin. In some embodiments, the biotin tag on the RNA binds to streptavidin. Streptavidin can be bound to a column, gel, film, or beads, the mixture containing the biotinylated molecule passed over streptavidin column, gel, film or beads in solution, and thereby purified away from non-biotinylated components.

In some embodiments, RNA is purified using a 5′ cap. Caps commonly consist of a terminal 7-methyl guanosine cap. However, common cap analogs are the Anti-Reverse Cap Analog (ARCA) 3′-O-Me-m⁷G(5′) ppp(5′)G, Standard Cap Analog m⁷G(5′)ppp(5′)G, Unmethylated Cap Analog G (5′)ppp(5′)G, Methylated Cap Analog for A+1 sites m⁷G(5′)ppp(5′)A, and the Unmethylated Cap Analog for A+1 sites G(5′)ppp(5′)A. The mRNA 5′ cap is bound by the EIF4E cap binding protein. EIF4E can in turn be bound to a gel, film, beads, or column over which the RNA mixture can be passed to purify the capped component.

In some embodiments the RNA can be purified using a poly(A) sequence in the RNA. In some embodiments, the poly(A) in the RNA binds to a poly(T) oligonucleotide bound to a gel, film, beads, or column. The poly(A) in the RNA basepairs with the poly(T), allowing for the purification of the RNA. This hybridization is reverse by changes in salt, pH, or temperature, allowing for purified poly(A) RNA to be eluted.

In some embodiments, the RNA is purified using an MS2 protein binding sequence. There may be one or copies of the MS2 protein binding sequence, typically in the untranslated regions of the RNA. MS2 protein, frequently fused to maltose binding protein, is conjugated to a gel, film, beads, or resin and the RNA mixture to be purified is passed over the gel, film, beads, or resin. The RNA-MS2 complex is then eluted by adding maltose.

In some embodiments, the RNA is purified using an aptamer sequence in the RNA. The aptamer has been artificially selected in vitro to bind to an additional molecule. Example aptamers are the D8 aptamer, which binds to Sephadex, or the 51 aptamer, which binds to Streptavidin. The RNA mixture is passed over a Sephadex or Streptavidin containing, and the RNA containing the aptamer binds to the matrix.

### Polynucleotides and Nucleic Acids

The term “polynucleotide,” in its broadest sense, includes any compound and/or substance that is or can be incorporated into an oligonucleotide chain. Exemplary polynucleotides suitable for the methods of present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, a polynucleotide suitable for the methods of the disclosure is an RNA. RNAs suitable for the methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof. In certain embodiments, the RNA is an mRNA.

Nucleic acids and polynucleotides suitable for the methods of the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a nucleic acid or polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide or nucleic acid (e.g., an mRNA) may include a 5′ cap structure, a chain terminating nucleotide, a stem loop, a polyA sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine or 1-ethyl-pseudouridine), and/or a 5-substituted cytidine (e.g., 5-methyl-cytidine).

Generally, the shortest length of a polynucleotide can be the length of the polynucleotide sequence that is sufficient to encode for a dipeptide. In another embodiment, the length of the polynucleotide sequence is sufficient to encode for a tripeptide. In another embodiment, the length of the polynucleotide sequence is sufficient to encode for a tetrapeptide. In another embodiment, the length of the polynucleotide sequence is sufficient to encode for a pentapeptide. In another embodiment, the length of the polynucleotide sequence is sufficient to encode for a hexapeptide. In another embodiment, the length of the polynucleotide sequence is sufficient to encode for a heptapeptide. In another embodiment, the length of the polynucleotide sequence is sufficient to encode for an octapeptide. In another embodiment, the length of the polynucleotide sequence is sufficient to encode for a nonapeptide. In another embodiment, the length of the polynucleotide sequence is sufficient to encode for a decapeptide.

Examples of dipeptides that the alternative polynucleotide sequences can encode for include, but are not limited to, carnosine and anserine.

In some cases, a polynucleotide is greater than 30 nucleotides in length. In another embodiment, the polynucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

Nucleic acids and polynucleotides may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

Nucleic acids and polynucleotides may include one or more alternative components, as described herein, which impart useful properties including increased stability and/or the lack of a substantial induction of the innate immune response of a cell into which the polynucleotide is introduced. For example, an alternative polynucleotide or nucleic acid exhibits reduced degradation in a cell into which the polynucleotide or nucleic acid is introduced, relative to a corresponding unaltered polynucleotide or nucleic acid. These alternative species may enhance the efficiency of protein production, intracellular retention of the polynucleotides, and/or viability of contacted cells, as well as possess reduced immunogenicity.

Polynucleotides and nucleic acids may be naturally or non-naturally occurring. Polynucleotides and nucleic acids may include one or more modified (e.g., altered or alternative) nucleobases, nucleosides, nucleotides, or combinations thereof. The nucleic acids and polynucleotides useful in the nanoparticle compositions described herein can include any useful modification or alteration, such as to the nucleobase, the sugar, or the internucleotide linkage (e.g., to a linking phosphate/to a phosphodiester linkage/to the phosphodiester backbone). In certain embodiments, alterations (e.g., one or more alterations) are present in each of the nucleobase, the sugar, and the internucleotide linkage. Alterations according to the present disclosure may be alterations of ribonucleic acids (RNAs) to deoxyribonucleic acids (DNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

Polynucleotides and nucleic acids may or may not be uniformly altered along the entire length of the molecule. For example, one or more or all types of nucleotide (e.g., purine or pyrimidine, or any one or more or all of A, G, U, C) may or may not be uniformly altered in a polynucleotide or nucleic acid, or in a given predetermined sequence region thereof. In some instances, all nucleotides X in a polynucleotide (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleotide linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure, or can be replaced by a plurality of compounds having different structures (e.g., 2, 3, 4, or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure, or can be replaced by a plurality of compounds having different structures (e.g., 2, 3, 4, or more unique structures).

The nucleic acids can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, and vectors). In some embodiments, the nucleic acids may include one or more messenger RNAs (mRNAs) having one or more alternative nucleoside or nucleotides (i.e., alternative mRNA molecules).

In some embodiments, a nucleic acid (e.g. mRNA) molecule, formula, composition or method associated therewith comprises one or more polynucleotides comprising features as described in WO2002/098443, WO2003/051401, WO2008/052770, WO2009127230, WO2006122828, WO2008/083949, WO2010088927, WO2010/037539, WO2004/004743, WO2005/016376, WO2006/024518, WO2007/095976, WO2008/014979, WO2008/077592, WO2009/030481, WO2009/095226, WO2011069586, WO2011026641, WO2011/144358, WO2012019780, WO2012013326, WO2012089338, WO2012113513, WO2012116811, WO2012116810, WO2013113502, WO2013113501, WO2013113736, WO2013143698, WO2013143699, WO2013143700, WO2013/120626, WO2013120627, WO2013120628, WO2013120629, WO2013174409, WO2014127917, WO2015/024669, WO2015/024668, WO2015/024667, WO2015/024665, WO2015/024666, WO2015/024664, WO2015101415, WO2015101414, WO2015024667, WO2015062738, WO2015101416, all of which are incorporated by reference herein.

### Nucleobase Alternatives

The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(τm⁵s²U), 1-taurinomethyl-4-thio-pseudouridine, 5-methyl-uracil (m⁵U, i.e., having the nucleobase deoxythymine), 1-methyl-pseudouridine (m¹ψ), 1-ethyl-pseudouridine (Et¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ ψ), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQ0), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

### Alterations on the Sugar

Nucleosides include a sugar molecule (e.g., a 5-carbon or 6-carbon sugar, such as pentose, ribose, arabinose, xylose, glucose, galactose, or a deoxy derivative thereof) in combination with a nucleobase, while nucleotides are nucleosides containing a nucleoside and a phosphate group or alternative group (e.g., boranophosphate, thiophosphate, selenophosphate, phosphonate, alkyl group, amidate, and glycerol). A nucleoside or nucleotide may be a canonical species, e.g., a nucleoside or nucleotide including a canonical nucleobase, sugar, and, in the case of nucleotides, a phosphate group, or may be an alternative nucleoside or nucleotide including one or more alternative components. For example, alternative nucleosides and nucleotides can be altered on the sugar of the nucleoside or nucleotide. In some embodiments, the alternative nucleosides or nucleotides include the structure:

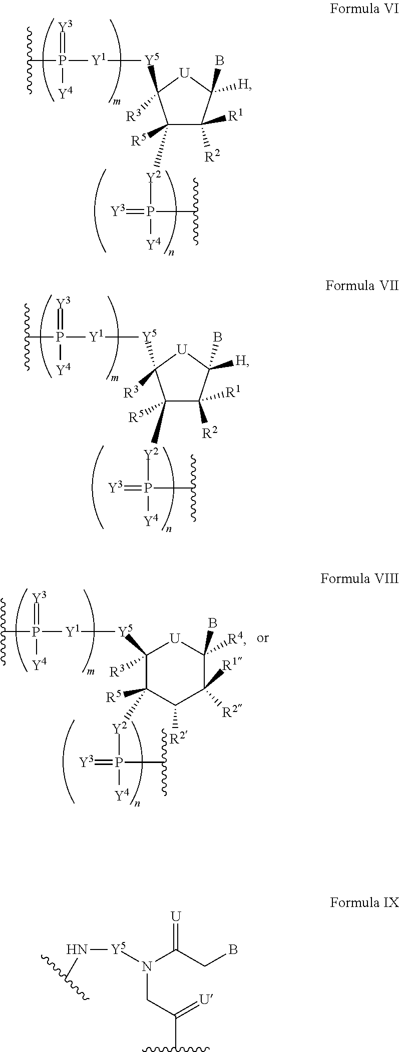

In each of the Formulae VI, VII, VIII, and IX, each of m and n is independently, an integer from 0 to 5, each of U and U′ independently, is O, S, N(R^{U})ₙᵤ, or C(R^{U})ₙᵤ, wherein nu is an integer from 0 to 2 and each R^{U} is, independently, H, halo, or optionally substituted alkyl;

each of R^{1′}, R^{2′}, R^{1″}, R^{2″}, R¹, R², R³, R⁴, and R⁵ is, independently, if present, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent; wherein the combination of R³ with one or more of R^{1′}, R^{1″}, R^{2′}, R^{2″}, or R⁵ (e.g., the combination of R^{1′} and R³, the combination of R^{1″} and R³, the combination of R^{2′} and R³, the combination of R^{2″} and R³, or the combination of R⁵ and R³) can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl); wherein the combination of R⁵ with one or more of R^{1′}, R^{2′}, or R²″ (e.g., the combination of and R⁵, the combination of and R⁵, the combination of R^{2′} and R⁵, or the combination of R^{2″} and R⁵) can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl); and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl); each of m′ and m″ is, independently, an integer from 0 to 3 (e.g., from 0 to 2, from 0 to 1, from 1 to 3, or from 1 to 2);

each of Y¹, Y², and Y³, is, independently, O, S, Se, —NR^{N1}—, optionally substituted alkylene, or optionally substituted heteroalkylene, wherein RN¹ is H, optionally substituted alkyl, optionally substituted alkenyl, optionally substituted alkynyl, optionally substituted aryl, or absent;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;

each Y⁵ is, independently, O, S, Se, optionally substituted alkylene (e.g., methylene), or optionally substituted heteroalkylene; and

B is a nucleobase, either modified or unmodified. In some embodiments, the 2′-hydroxy group (OH) can be modified or replaced with a number of different substituents. Exemplary substitutions at the 2′-position include, but are not limited to, H, azido, halo (e.g., fluoro), optionally substituted C₁₋₆ alkyl (e.g., methyl); optionally substituted C₁₋₆ alkoxy (e.g., methoxy or ethoxy); optionally substituted C₆₋₁₀ aryloxy; optionally substituted C₃₋₈ cycloalkyl; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxy is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a polynucleotide molecule can include nucleotides containing, e.g., arabinose or L-ribose, as the sugar.

In some embodiments, the polynucleotide includes at least one nucleoside wherein the sugar is L-ribose, 2′-O-methyl-ribose, 2′-fluoro-ribose, arabinose, hexitol, an LNA, or a PNA.

### Alterations on the Internucleotide Linkage

Alternative nucleotides can be altered on the internucleotide linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleotides can include the wholesale replacement of an unaltered phosphate moiety with another internucleotide linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy.

The replacement of one or more of the oxygen atoms at the a position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleotide linkages that may be employed according to the present disclosure, including internucleotide linkages which do not contain a phosphorous atom, are described herein.

### Internal Ribosome Entry Sites

Polynucleotides may contain an internal ribosome entry site (IRES). An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### 5′-Cap Structure

A polynucleotide (e.g., an mRNA) may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional alternative guanosine nucleotides may be used such as α-methyl-phosphonate and seleno-phosphate nucleotides.

Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule.

5′-Cap structures include those described in International Patent Publication Nos. WO2008127688, WO 2008016473, and WO 2011015347, the cap structures of each of which are incorporated herein by reference.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7, 3′-O-dimethyl-guanosine-5′-triphosphate-5′-guanosine, m⁷G-3′mppp-G, which may equivalently be designated 3′ O-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methlyated guanosine provides the terminal moiety of the capped polynucleotide (e.g., mRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxyethyl) substituted dinucleotide cap analog known in the art and/or described herein. Non-limiting examples of N7-(4-chlorophenoxyethyl) substituted dinucleotide cap analogs include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxyethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap 1 structure. This cap results in a higher translational-competency, cellular stability, and a reduced activation of cellular pro-inflammatory cytokines, as compared, e.g., to other 5′cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the alternative polynucleotides may be capped. This is in contrast to ˜80% when a cap analog is linked to a polynucleotide in the course of an in vitro transcription reaction.

5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine.

In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

### 5′-UTRs

A 5′-UTR may be provided as a flanking region to polynucleotides (e.g., mRNAs). A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

Shown in Table 21 in U.S. Provisional Application No. 61/775,509, and in Table 21 and in Table 22 in U.S. Provisional Application No. 61/829,372, of which are incorporated herein by reference, is a listing of the start and stop site of alternative polynucleotides (e.g., mRNA). In Table 21 each 5′-UTR (5′-UTR-005 to 5′-UTR 68511) is identified by its start and stop site relative to its native or wild type (homologous) transcript (ENST; the identifier used in the ENSEMBL database).

To alter one or more properties of a polynucleotide (e.g., mRNA), 5′-UTRs which are heterologous to the coding region of an alternative polynucleotide (e.g., mRNA) may be engineered. The polynucleotides (e.g., mRNA) may then be administered to cells, tissue or organisms and outcomes such as protein level, localization, and/or half-life may be measured to evaluate the beneficial effects the heterologous 5′-UTR may have on the alternative polynucleotides (mRNA). Variants of the 5′-UTRs may be utilized wherein one or more nucleotides are added or removed to the termini, including A, T, C, or G. 5′-UTRs may also be codon-optimized, or altered in any manner described herein.

### 5′-UTRs, 3′-UTRs, and Translation Enhancer Elements (TEES)

The 5′-UTR of a polynucleotides (e.g., mRNA) may include at least one translation enhancer element. The term “translational enhancer element” refers to sequences that increase the amount of polypeptide or protein produced from a polynucleotide. As a non-limiting example, the TEE may be located between the transcription promoter and the start codon. The polynucleotides (e.g., mRNA) with at least one TEE in the 5′-UTR may include a cap at the 5′-UTR. Further, at least one TEE may be located in the 5′-UTR of polynucleotides (e.g., mRNA) undergoing cap-dependent or cap-independent translation.

In one aspect, TEEs are conserved elements in the UTR which can promote translational activity of a polynucleotide such as, but not limited to, cap-dependent or cap-independent translation. The conservation of these sequences has been previously shown by Panek et al. (Nucleic Acids Research, 2013, 1-10) across 14 species including humans.

In one non-limiting example, the TEEs known may be in the 5′-leader of the Gtx homeodomain protein (Chappell et al., Proc. Natl. Acad. Sci. USA 101:9590-9594, 2004, the TEEs of which are incorporated herein by reference).

In another non-limiting example, TEEs are disclosed in US Patent Publication Nos. 2009/0226470 and 2013/0177581, International Patent Publication Nos. WO2009/075886, WO2012/009644, and WO1999/024595, U.S. Pat. Nos. 6,310,197 and 6,849,405, the TEE sequences disclosed in each of which are incorporated herein by reference.

In yet another non-limiting example, the TEE may be an internal ribosome entry site (IRES), HCV-IRES or an IRES element such as, but not limited to, those described in U.S. Pat. No. 7,468,275, US Patent Publication Nos. 2007/0048776 and 2011/0124100 and International Patent Publication Nos. WO2007/025008 and WO2001/055369, the IRES sequences of each of which are incorporated herein by reference. The IRES elements may include, but are not limited to, the Gtx sequences (e.g., Gtx9-nt, Gtx8-nt, Gtx7-nt) described by Chappell et al. (Proc. Natl. Acad. Sci. USA 101:9590-9594, 2004) and Zhou et al. (PNAS 102:6273-6278, 2005) and in US Patent Publication Nos. 2007/0048776 and 2011/0124100 and International Patent Publication No. WO2007/025008, the IRES sequences of each of which are incorporated herein by reference.

“Translational enhancer polynucleotides” are polynucleotides which include one or more of the specific TEE exemplified herein and/or disclosed in the art (see e.g., U.S. Pat. Nos. 6,310,197, 6,849,405, 7,456,273, 7,183,395, U.S. Patent Publication Nos. 20090/226470, 2007/0048776, 2011/0124100, 2009/0093049, 2013/0177581, International Patent Publication Nos. WO2009/075886, WO2007/025008, WO2012/009644, WO2001/055371, WO1999/024595, and European Patent Nos. 2610341 and 2610340; the TEE sequences of each of which are incorporated herein by reference) or their variants, homologs or functional derivatives. One or multiple copies of a specific TEE can be present in a polynucleotide (e.g., mRNA). The TEEs in the translational enhancer polynucleotides can be organized in one or more sequence segments. A sequence segment can harbor one or more of the specific TEEs exemplified herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the specific TEEs exemplified herein, identical, or different number of copies of each of the specific TEEs, and/or identical or different organization of the TEEs within each sequence segment.

A polynucleotide (e.g., mRNA) may include at least one TEE that is described in International Patent Publication Nos. WO1999/024595, WO2012/009644, WO2009/075886, WO2007/025008, WO1999/024595, European Patent Publication Nos. 2610341 and 2610340, U.S. Pat. Nos. 6,310,197, 6,849,405, 7,456,273, 7,183,395, and US Patent Publication Nos. 2009/0226470, 2011/0124100, 2007/0048776, 2009/0093049, and 2013/0177581 the TEE sequences of each of which are incorporated herein by reference. The TEE may be located in the 5′-UTR of the polynucleotides (e.g., mRNA).

A polynucleotide (e.g., mRNA) may include at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. 2009/0226470, 2007/0048776, 2013/0177581 and 2011/0124100, International Patent Publication Nos. WO1999/024595, WO2012/009644, WO2009/075886 and WO2007/025008, European Patent Publication Nos. 2610341 and 2610340, U.S. Pat. Nos. 6,310,197, 6,849,405, 7,456,273, 7,183,395, the TEE sequences of each of which are incorporated herein by reference.

The 5′-UTR of a polynucleotide (e.g., mRNA) may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 5′-UTR of a polynucleotide (e.g., mRNA) may be the same or different TEE sequences. The TEE sequences may be in a pattern such as ABABAB, AABBAABBAABB, or ABCABCABC, or variants thereof, repeated once, twice, or more than three times. In these patterns, each letter, A, B, or C represent a different TEE sequence at the nucleotide level.

In some cases, the 5′-UTR may include a spacer to separate two TEE sequences. As a non-limiting example, the spacer may be a 15 nucleotide spacer and/or other spacers known in the art. As another non-limiting example, the 5′-UTR may include a TEE sequence-spacer module repeated at least once, at least twice, at least 3 times, at least 4 times, at least 5 times, at least 6 times, at least 7 times, at least 8 times, at least 9 times, or more than 9 times in the 5′-UTR.

In other instances, the spacer separating two TEE sequences may include other sequences known in the art which may regulate the translation of the polynucleotides (e.g., mRNA) of the present disclosure such as, but not limited to, miR sequences (e.g., miR binding sites and miR seeds). As a non-limiting example, each spacer used to separate two TEE sequences may include a different miR sequence or component of a miR sequence (e.g., miR seed sequence).

In some instances, the TEE in the 5′-UTR of a polynucleotide (e.g., mRNA) may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in US Patent Publication Nos. 2009/0226470, 2007/0048776, 2013/0177581 and 2011/0124100, International Patent Publication Nos. WO1999/024595, WO2012/009644, WO2009/075886 and WO2007/025008, European Patent Publication Nos. 2610341 and 2610340, and U.S. Pat. Nos. 6,310,197, 6,849,405, 7,456,273, and 7,183,395 the TEE sequences of each of which are incorporated herein by reference. In another embodiment, the TEE in the 5′-UTR of the polynucleotides (e.g., mRNA) of the present disclosure may include a 5-30 nucleotide fragment, a 5-25 nucleotide fragment, a 5-20 nucleotide fragment, a 5-15 nucleotide fragment, a 5-10 nucleotide fragment of the TEE sequences disclosed in US Patent Publication Nos. 2009/0226470, 2007/0048776, 2013/0177581 and 2011/0124100, International Patent Publication Nos. WO1999/024595, WO2012/009644, WO2009/075886 and WO2007/025008, European Patent Publication Nos. 2610341 and 2610340, and U.S. Pat. Nos. 6,310,197, 6,849,405, 7,456,273, and 7,183,395; the TEE sequences of each of which are incorporated herein by reference.

In certain cases, the TEE in the 5′-UTR of the polynucleotides (e.g., mRNA) of the present disclosure may include at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more than 99% of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Genome-wide profiling of human cap-independent translation-enhancing elements, Nature Methods, 2013; DOI:10.1038/NMETH.2522); the TEE sequences of each of which are herein incorporated by reference. In another embodiment, the TEE in the 5′-UTR of the polynucleotides (e.g., mRNA) of the present disclosure may include a 5-30 nucleotide fragment, a 5-25 nucleotide fragment, a 5-20 nucleotide fragment, a 5-15 nucleotide fragment, a 5-10 nucleotide fragment of the TEE sequences disclosed in Chappell et al. (Proc. Natl. Acad. Sci. USA 101:9590-9594, 2004) and Zhou et al. (PNAS 102:6273-6278, 2005), in Supplemental Table 1 and in Supplemental Table 2 disclosed by Wellensiek et al (Genome-wide profiling of human cap-independent translation-enhancing elements, Nature Methods, 2013; DOI:10.1038/NMETH.2522); the TEE sequences of each of which is incorporated herein by reference.

In some cases, the TEE used in the 5′-UTR of a polynucleotide (e.g., mRNA) is an IRES sequence such as, but not limited to, those described in U.S. Pat. No. 7,468,275 and International Patent Publication No. WO2001/055369, the TEE sequences of each of which are incorporated herein by reference.

In some instances, the TEEs used in the 5′-UTR of a polynucleotide (e.g., mRNA) may be identified by the methods described in US Patent Publication Nos. 2007/0048776 and 2011/0124100 and International Patent Publication Nos. WO2007/025008 and WO2012/009644, the methods of each of which are incorporated herein by reference.

In some cases, the TEEs used in the 5′-UTR of a polynucleotide (e.g., mRNA) of the present disclosure may be a transcription regulatory element described in U.S. Pat. Nos. 7,456,273 and 7,183,395, US Patent Publication No. 2009/0093049, and International Publication No. WO2001/055371, the TEE sequences of each of which is incorporated herein by reference. The transcription regulatory elements may be identified by methods known in the art, such as, but not limited to, the methods described in U.S. Pat. Nos. 7,456,273 and 7,183,395, US Patent Publication No. 2009/0093049, and International Publication No. WO2001/055371, the methods of each of which is incorporated herein by reference.

In yet other instances, the TEE used in the 5′-UTR of a polynucleotide (e.g., mRNA) is a polynucleotide or portion thereof as described in U.S. Pat. Nos. 7,456,273 and 7,183,395, US Patent Publication No. 2009/0093049, and International Publication No. WO2001/055371, the TEE sequences of each of which are incorporated herein by reference.

The 5′-UTR including at least one TEE described herein may be incorporated in a monocistronic sequence such as, but not limited to, a vector system or a polynucleotide vector. As a non-limiting example, the vector systems and polynucleotide vectors may include those described in U.S. Pat. Nos. 7,456,273 and 7,183,395, US Patent Publication Nos. 2007/0048776, 2009/0093049 and 2011/0124100, and International Patent Publication Nos. WO2007/025008 and WO2001/055371, the TEE sequences of each of which are incorporated herein by reference.

The TEEs described herein may be located in the 5′-UTR and/or the 3′-UTR of the polynucleotides (e.g., mRNA). The TEEs located in the 3′-UTR may be the same and/or different than the TEEs located in and/or described for incorporation in the 5′-UTR.

In some cases, the 3′-UTR of a polynucleotide (e.g., mRNA) may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. The TEE sequences in the 3′-UTR of the polynucleotides (e.g., mRNA) of the present disclosure may be the same or different TEE sequences. The TEE sequences may be in a pattern such as ABABAB, AABBAABBAABB, or ABCABCABC, or variants thereof, repeated once, twice, or more than three times. In these patterns, each letter, A, B, or C represent a different TEE sequence at the nucleotide level.

In one instance, the 3′-UTR may include a spacer to separate two TEE sequences. As a non-limiting example, the spacer may be a 15 nucleotide spacer and/or other spacers known in the art. As another non-limiting example, the 3′-UTR may include a TEE sequence-spacer module repeated at least once, at least twice, at least 3 times, at least 4 times, at least 5 times, at least 6 times, at least 7 times, at least 8 times, at least 9 times, or more than 9 times in the 3′-UTR.

In other cases, the spacer separating two TEE sequences may include other sequences known in the art which may regulate the translation of the polynucleotides (e.g., mRNA) of the present disclosure such as, but not limited to, miR sequences described herein (e.g., miR binding sites and miR seeds). As a non-limiting example, each spacer used to separate two TEE sequences may include a different miR sequence or component of a miR sequence (e.g., miR seed sequence).

In yet other cases, the incorporation of a miR sequence and/or a TEE sequence changes the shape of the stem loop region which may increase and/or decrease translation. (See e.g., Kedde et al. A Pumilio-induced RNA structure switch in p27-3′UTR controls miR-221 and miR-22 accessibility. Nature Cell Biology. 2010).

### Stem Loops

Polynucleotides (e.g., mRNAs) may include a stem loop such as, but not limited to, a histone stem loop. The stem loop may be a nucleotide sequence that is about 25 or about 26 nucleotides in length such as, but not limited to, those as described in International Patent Publication No. WO2013/103659, which is incorporated herein by reference. The histone stem loop may be located 3′-relative to the coding region (e.g., at the 3′-terminus of the coding region). As a non-limiting example, the stem loop may be located at the 3′-end of a polynucleotide described herein. In some cases, a polynucleotide (e.g., an mRNA) includes more than one stem loop (e.g., two stem loops). Examples of stem loop sequences are described in International Patent Publication Nos. WO2012/019780 and WO201502667, the stem loop sequences of which are herein incorporated by reference. In some instances, a polynucleotide includes the stem loop sequence CAAAGGCTCTTTTCAGAGCCACCA (SEQ ID NO: 1). In other instances, a polynucleotide includes the stem loop sequence CAAAGGCUCUUUUCAGAGCCACCA (SEQ ID NO: 2).

A stem loop may be located in a second terminal region of a polynucleotide. As a non-limiting example, the stem loop may be located within an untranslated region (e.g., 3′-UTR) in a second terminal region.

In some cases, a polynucleotide such as, but not limited to mRNA, which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide.

In other cases, a polynucleotide such as, but not limited to mRNA, which includes the histone stem loop may be stabilized by an alteration to the 3′-region of the polynucleotide that can prevent and/or inhibit the addition of oligio(U) (see e.g., International Patent Publication No. WO2013/103659).

In yet other cases, a polynucleotide such as, but not limited to mRNA, which includes the histone stem loop may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

In some instances, the polynucleotides of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein.

In other instances, the polynucleotides of the present disclosure may include a histone stem loop and a 5′-cap structure. The 5′-cap structure may include, but is not limited to, those described herein and/or known in the art.

In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

In certain instances, the conserved stem loop region may include a miR sequence described herein and may also include a TEE sequence.

In some cases, the incorporation of a miR sequence and/or a TEE sequence changes the shape of the stem loop region which may increase and/or decrease translation. (See e.g., Kedde et al. A Pumilio-induced RNA structure switch in p27-3′UTR controls miR-221 and miR-22 accessibility. Nature Cell Biology. 2010, herein incorporated by reference in its entirety).

Polynucleotides may include at least one histone stem-loop and a poly-A region or polyadenylation signal. Non-limiting examples of polynucleotide sequences encoding for at least one histone stem-loop and a poly-A region or a polyadenylation signal are described in International Patent Publication No. WO2013/120497, WO2013/120629, WO2013/120500, WO2013/120627, WO2013/120498, WO2013/120626, WO2013/120499, and WO2013/120628, the sequences of each of which are incorporated herein by reference. In certain cases, the polynucleotide encoding for a histone stem loop and a poly-A region or a polyadenylation signal may code for a pathogen antigen or fragment thereof such as the polynucleotide sequences described in International Patent Publication No WO2013/120499 and WO2013/120628, the sequences of both of which are incorporated herein by reference. In other cases, the polynucleotide encoding for a histone stem loop and a poly-A region or a polyadenylation signal may code for a therapeutic protein such as the polynucleotide sequences described in International Patent Publication No WO2013/120497 and WO2013/120629, the sequences of both of which are incorporated herein by reference. In some cases, the polynucleotide encoding for a histone stem loop and a poly-A region or a polyadenylation signal may code for a tumor antigen or fragment thereof such as the polynucleotide sequences described in International Patent Publication No WO2013/120500 and WO2013/120627, the sequences of both of which are incorporated herein by reference. In other cases, the polynucleotide encoding for a histone stem loop and a poly-A region or a polyadenylation signal may code for an allergenic antigen or an autoimmune self-antigen such as the polynucleotide sequences described in International Patent Publication No WO2013/120498 and WO2013/120626, the sequences of both of which are incorporated herein by reference.

### Poly-A Regions

A polynucleotide or nucleic acid (e.g., an mRNA) may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid.

During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long.

Unique poly-A region lengths may provide certain advantages to the alternative polynucleotides of the present disclosure.

Generally, the length of a poly-A region of polynucleotides of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides.

In some instances, the poly-A region may be 80 nucleotides, 120 nucleotides, 160 nucleotides in length on an alternative polynucleotide molecule described herein.

In other instances, the poly-A region may be 20, 40, 80, 100, 120, 140, or 160 nucleotides in length on an alternative polynucleotide molecule described herein.

In some cases, the poly-A region is designed relative to the length of the overall alternative polynucleotide. This design may be based on the length of the coding region of the alternative polynucleotide, the length of a particular feature or region of the alternative polynucleotide (such as mRNA), or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of polynucleotides (e.g., mRNA) for poly-A binding protein may be used to enhance expression. The engineered binding sites may be sensor sequences which can operate as binding sites for ligands of the local microenvironment of the polynucleotides (e.g., mRNA). As a non-limiting example, the polynucleotides (e.g., mRNA) may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct polynucleotides (e.g., mRNA) may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site.

In certain cases, a poly-A region may be used to modulate translation initiation. While not wishing to be bound by theory, the poly-A region recruits PABP which in turn can interact with translation initiation complex and thus may be essential for protein synthesis.

In some cases, a poly-A region may also be used in the present disclosure to protect against 3′-5′-exonuclease digestion.

In some instances, a polynucleotide (e.g., mRNA) may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet results in protein production equivalent to at least 75% of that seen using a poly-A region of 120 nucleotides alone.

In some cases, a polynucleotide (e.g., mRNA) may include a poly-A region and may be stabilized by the addition of a 3′-stabilizing region. The polynucleotides (e.g., mRNA) with a poly-A region may further include a 5′-cap structure.

In other cases, a polynucleotide (e.g., mRNA) may include a poly-A-G Quartet. The polynucleotides (e.g., mRNA) with a poly-A-G Quartet may further include a 5′-cap structure.

In some cases, the 3′-stabilizing region which may be used to stabilize a polynucleotide (e.g., mRNA) including a poly-A region or poly-A-G Quartet may be, but is not limited to, those described in International Patent Publication No. WO2013/103659, the poly-A regions and poly-A-G Quartets of which are incorporated herein by reference. In other cases, the 3′-stabilizing region which may be used with the polynucleotides of the present disclosure include a chain termination nucleoside such as 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside.

In other cases, a polynucleotide such as, but not limited to mRNA, which includes a polyA region or a poly-A-G Quartet may be stabilized by an alteration to the 3′-region of the polynucleotide that can prevent and/or inhibit the addition of oligio(U) (see e.g., International Patent Publication No. WO2013/103659).

In yet other instances, a polynucleotide such as, but not limited to mRNA, which includes a poly-A region or a poly-A-G Quartet may be stabilized by the addition of an oligonucleotide that terminates in a 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

### Chain Terminating Nucleosides

A nucleic acid may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

As used herein, “alkyl” is intended to include straight chain (linear) saturated aliphatic hydrocarbon groups and branched saturated aliphatic hydrocarbon groups. In some embodiments, is intended to include C₁, C₂, C₃, C₄, C₅ or C₆ alkyl groups. Examples of alkyl include moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety (i.e., aryl or heteroaryl).

Optionally substituted alkenyl “Alkenyl” includes unsaturated aliphatic groups analogous in length and possible substitution to the alkyls described above, but that contain at least one double bond. In some embodiments, the term “alkenyl” includes straight chain alkenyl groups (e.g., ethenyl, propenyl, butenyl, pentenyl, hexenyl, heptenyl, octenyl, nonenyl, decenyl), and branched alkenyl groups.

In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, cycloalkyl, heterocyclyl, alkylaryl, or an aromatic or heteroaromatic moiety (i.e., aryl or heteroaryl).

“Alkynyl” includes unsaturated aliphatic groups analogous in length and possible substitution to the alkyls described above, but which contain at least one triple bond. In some embodiments, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety (i.e., aryl or heteroaryl).

As used herein, “amine” or “amino” refers to —NH₂. Amino groups may be further substituted so as to include, e.g. alkylamino, dialkylamino, arylamino, diarylamino and alkylarylamino. “Alkylamino” includes groups of compounds wherein the nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to two alkyl groups. Examples of dialkylamino groups include, but are not limited to, dimethylamino and diethylamino. “Acylamino” and “diarylamino” include groups wherein the nitrogen is bound to at least one or two aryl groups, respectively. “Aminoaryl” and “aminoaryloxy” refer to aryl and aryloxyl substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group.

As used herein, the term “thiol” refers to —SH. Thiol groups may be further substituted so as to include, e.g. alkylthio, alkenylthio, sulfonyl, alkylsulfonyl, sulfinyl, alkylsulfinyl or sulfamoyl. “Alkylthio” includes groups of compounds wherein the H of SH is replaced by an alkyl group. Examples of alkylamino groups include benzylazido, methylazido, ethylazido, phenethylaazido, etc. “Sulfonyl” refers to groups wherein the sulfur atom is connected with double bonds to two oxygen atoms. “Alkylsulfonyl” includes compounds and moieties which contain an alkyl group connected with a single bond to a sulfonyl group. “Sulfinyl” refers to groups wherein the sulfur atom is connected with double bonds to one oxygen atom. “Alkylsulfinyl” includes compounds and moieties which contain an alkyl group connected with a single bond to a sulfinyl group. “Sulfamoyl” includes compounds and moieties which contain an amino group connected with a single bond to a sulfonyl group.

As used herein, “azido” refers to —N₃. Azido groups may be further substituted so as to include, e.g. alkylazido, dialkylazido, alkenylazido, dialkenylazido, alkylalkenylazido, arylazido, diarylazido, alkylarylazido and arylalkenylazido. “Alkylazido” includes groups of compounds wherein one of the nitrogen of —N₃ is bound to at least one alkyl group. Examples of alkylamino groups include benzylazido, methylazido, ethylazido, phenethylaazido, etc. “Dialkylamino” includes groups wherein the nitrogen of —N₃ is bound to two alkyl groups. Examples of dialkylamino groups include, but are not limited to, dimethylazido and diethylazido. “Acylamino” and “diarylamino” include groups wherein the nitrogen is bound to at least one or two aryl groups, respectively.

As used herein, “halo” or “halogen” refers to fluoro, chloro, bromo and iodo.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

The term “optionally substituted,” as used herein, means not being substituted (e.g., none of the one or more hydrogen atoms on the designated variable is replaced with any other group) or being substituted (e.g., any one or more hydrogen atoms on the designated variable is replaced with a suitable group, provided that the designated atom's normal valency is not exceeded, and that the substitution results in a stable compound).

Any of the substituents on compounds or moieties defined herein may be further substituted as described herein for the compounds or moieties constituting those substituents. For example, an alkyl substituent on any group can be “substituted alkyl” as described herein.

About, Approximately: As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

Compound: As used herein, the term “compound,” is meant to include all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

Delivering: As used herein, the term “delivering” means providing an entity to a destination. For example, delivering a therapeutic and/or prophylactic agent to a subject may involve administering a nanoparticle composition including the therapeutic and/or prophylactic agent to the subject (e.g., by an intravenous, intramuscular, intradermal, or subcutaneous route). Administration of a nanoparticle composition to a mammal or mammalian cell may involve contacting one or more cells with the nanoparticle composition.

Enhanced delivery: As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic agent by a nanoparticle to a target tissue of interest (e.g., mammalian liver) compared to the level of delivery of a therapeutic and/or prophylactic agent by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic agent in a tissue to the weight of said tissue, comparing the amount of protein produced in a tissue to the amount of total protein in said tissue, or comparing the amount of therapeutic and/or prophylactic agent in a tissue to the amount of total therapeutic and/or prophylactic agent in said tissue. It will be understood that the enhanced delivery of a nanoparticle to a target tissue need not be determined in a subject being treated, it may be determined in a surrogate such as an animal model (e.g., a rat model).

Specific delivery: As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic agent by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic agent in a tissue to the weight of said tissue, comparing the amount of protein produced in a tissue to the amount of total protein in said tissue, or comparing the amount of therapeutic and/or prophylactic agent in a tissue to the amount of total therapeutic and/or prophylactic agent in said tissue. For example, for renovascular targeting, a therapeutic and/or prophylactic agent is specifically provided to a mammalian kidney as compared to the liver and spleen if 1.5, 2-fold, 3-fold, 5-fold, 10-fold, 15 fold, or 20 fold more therapeutic and/or prophylactic agent per 1 g of tissue is delivered to a kidney compared to that delivered to the liver or spleen following systemic administration of the therapeutic and/or prophylactic agent. It will be understood that the ability of a nanoparticle to specifically deliver to a target tissue need not be determined in a subject being treated, it may be determined in a surrogate such as an animal model (e.g., a rat model).

Encapsulation efficiency: As used herein, “encapsulation efficiency” refers to the amount of a therapeutic and/or prophylactic agent that becomes part of a nanoparticle composition, relative to the initial total amount of therapeutic and/or prophylactic agent used in the preparation of a nanoparticle composition. For example, if 97 mg of therapeutic and/or prophylactic agent are encapsulated in a nanoparticle composition out of a total 100 mg of therapeutic and/or prophylactic agent initially provided to the composition, the encapsulation efficiency may be given as 97%. As used herein, “encapsulation” may refer to complete, substantial, or partial enclosure, confinement, surrounding, or encasement.

Expression: As used herein, “expression” of a nucleic acid sequence refers to translation of an mRNA into a polypeptide or protein and/or post-translational modification of a polypeptide or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Isomer: As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

Lipid component: As used herein, a “lipid component” is that component of a nanoparticle composition that includes one or more lipids. For example, the lipid component may include one or more cationic/ionizable, PEGylated, structural, or other lipids, such as phospholipids.

Linker: As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

Methods of administration: As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

Modified: As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

N:P ratio: As used herein, the “N:P ratio” is the molar ratio of ionizable (in the physiological pH range) nitrogen atoms in a lipid to phosphate groups in an RNA, e.g., in a nanoparticle composition including a lipid component and an RNA.

Nanoparticle composition: As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

Naturally occurring: As used herein, “naturally occurring” means existing in nature without artificial aid.

Patient: As used herein, “patient” refers to a subject who may seek or be in need of treatment, requires treatment, is receiving treatment, will receive treatment, or a subject who is under care by a trained professional for a particular disease or condition.

PEG lipid: As used herein, a “PEG lipid” or “PEGylated lipid” refers to a lipid comprising a polyethylene glycol component.

Pharmaceutically acceptable: The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability, and solubility. Recrystallization solvent, rate of crystallization, storage temperature, and other factors may cause one crystal form to dominate. Crystal polymorphs of the compounds can be prepared by crystallization under different conditions.

Pharmaceutically acceptable salts: Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Phospholipid: As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

Polydispersity index: As used herein, the “polydispersity index” is a ratio that describes the homogeneity of the particle size distribution of a system. A small value, e.g., less than 0.3, indicates a narrow particle size distribution.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

Single unit dose: As used herein, a “single unit dose” is a dose of any therapeutic administered in one dose/at one time/single route/single point of contact, i.e., single administration event.

Split dose: As used herein, a “split dose” is the division of single unit dose or total daily dose into two or more doses.

Total daily dose: As used herein, a “total daily dose” is an amount given or prescribed in 24 hour period. It may be administered as a single unit dose.

Size: As used herein, “size” or “mean size” in the context of nanoparticle compositions refers to the mean diameter of a nanoparticle composition.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human, and most preferably a patient.

Target tissue: As used herein “target tissue” refers to any one or more tissue types of interest in which the delivery of a therapeutic and/or prophylactic agent would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

Therapeutic and/or prophylactic agent: The term “therapeutic agent” refers to any agent that, when administered to a subject, has a therapeutic and/or diagnostic effect and/or elicits a desired biological and/or pharmacological effect. The term “prophylactic agent” refers to any agent that, when administered to a subject, has a prophylactic effect. Therapeutic and/or prophylactic agents are also referred to as “actives” or “active agents.” Such agents include, but are not limited to, cytotoxins, radioactive ions, chemotherapeutic agents, small molecule drugs, proteins, and nucleic acids.

Therapeutically effective amount: As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Transfection: As used herein, “transfection” refers to the introduction of a species (e.g., an RNA) into a cell. Transfection may occur, for example, in vitro, ex vivo, or in vivo.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Zeta potential: As used herein, the “zeta potential” is the electrokinetic potential of a lipid e.g., in a particle composition.

Those skilled in the art will recognize, or be able to ascertain using no more than routine experimentation, many equivalents to the specific embodiments in accordance with the present disclosure. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process. As used herein, the expressions “one or more of A, B, or C,” “one or more A, B, or C,” “one or more of A, B, and C,” “one or more A, B, and C”, “selected from A, B, and C,” “selected from the group consisting of A, B, and C,” and the like are used interchangeably and all refer to a selection from a group consisting of A, B, and/or C, i.e., one or more As, one or more Bs, one or more Cs, or any combination thereof, unless otherwise specified.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

### Example 1—Ligation of a Dinucleotide Acceptors onto a Chemically Synthesized RNA Donor

A dinucleotide acceptor with a 5′-triphosphate group (1) was ligated onto a chemically synthesized RNA donor (SEQ ID No.: 3: 5′-P-rGrArGrUrArArGrArArGrArArArUrArUrArArGrArGrCrCrArCrC-Cyanine3-3′):

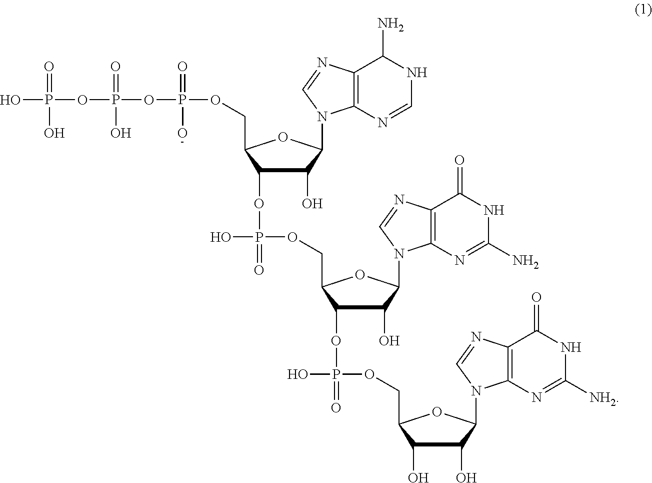

A dinucleotide acceptor with a 5′-triphosphate group (3) was ligated onto a chemically synthesized RNA donor (SEQ ID No.: 3):

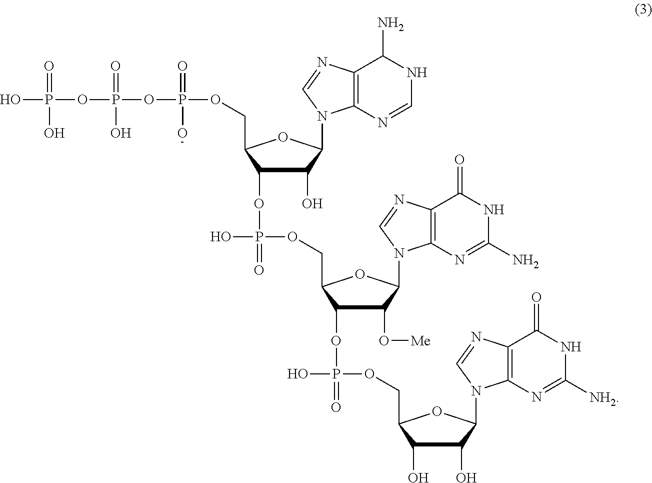

A dinucleotide acceptor with 5′-inverted guanosine (4) was ligated onto a chemically synthesized RNA donor (SEQ ID No.: 3):

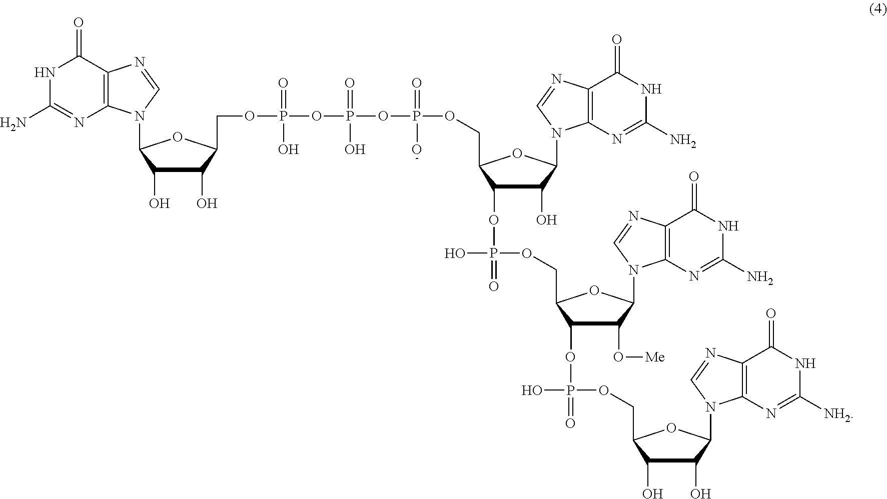

### Example 2: Ligation Using T4 DNA Ligase

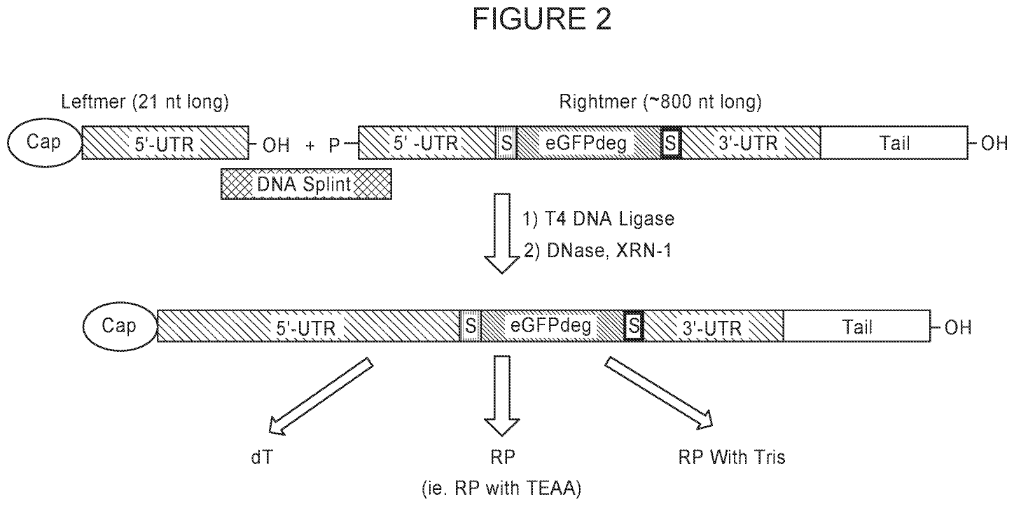

An mRNA was produced by ligation with T4 DNA ligase and a splint, and tested for B-cell activation and expression in black mice. An acceptor moiety 21 nucleotides in length and comprising a cap, a 5′-untranslated region and a 3′ OH group (leftmer) was ligated to the 5′ triphosphate group of a donor moiety, about 800 nucleotides long and (rightmer). The rightmer feedstocks were purified by either oligo(dT) purification or reverse phase chromatography. Following ligation, the splint was removed using DNase and the unreacted rightmer was removed with XRN-1. The ligation product was purified by different methods (i.e., oligo(dT) purification, reverse phase chromatography with triethylammonium acetate, or reverse phase chromatography with tris(hydroxymethyl)aminomethane). The reaction was conducted on a 4 mg scale (FIG. 2).

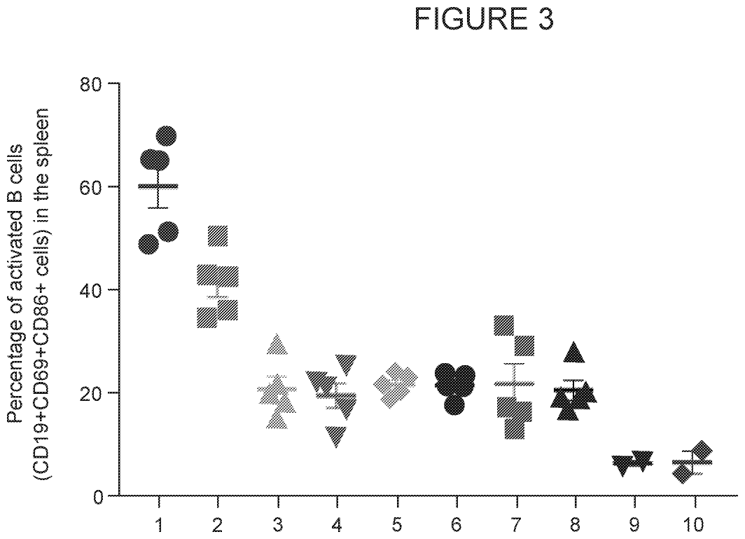

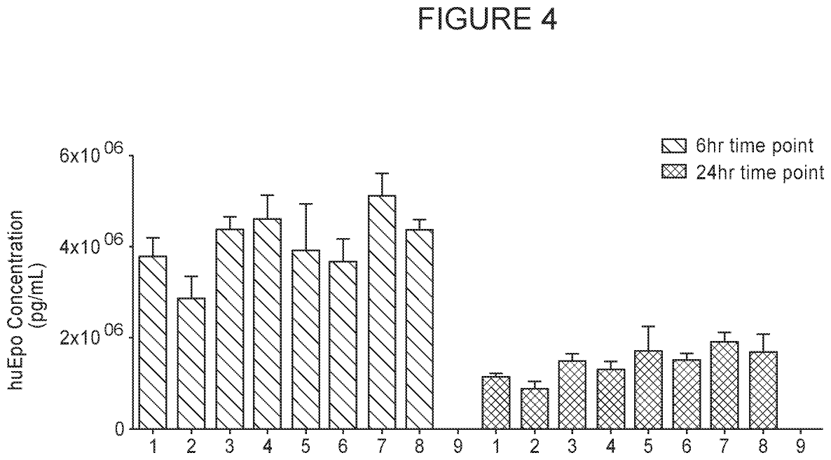

mRNAs produced via the ligation method, employing various purification methods for the product and the feedstock were tested for B-cell activation and huEpo expression in 6 black mice. The results are summarized in FIGS. 3 and 4, respectively.

### Example 3: Installation of Modified 5′-Untranslated Regions

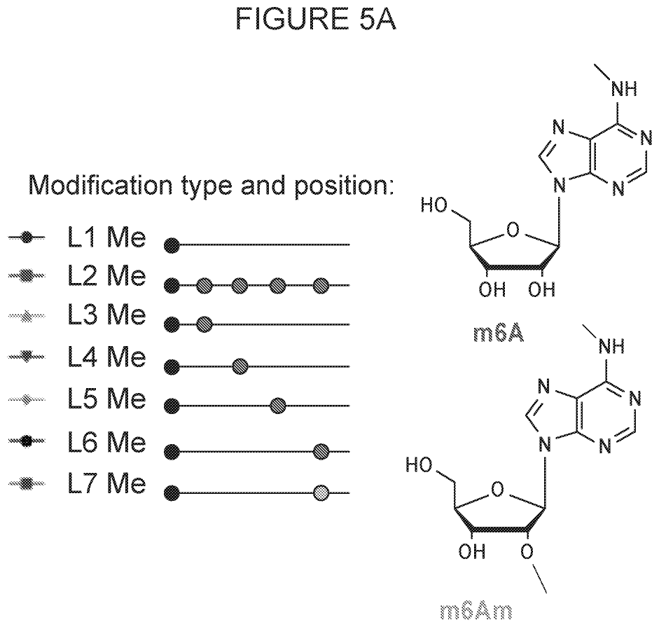

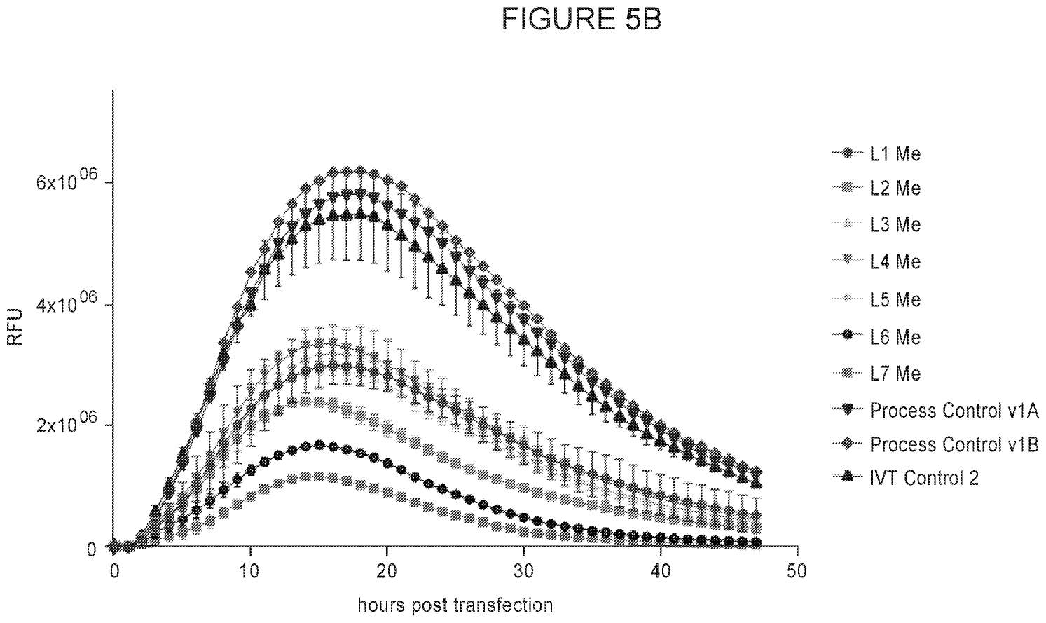

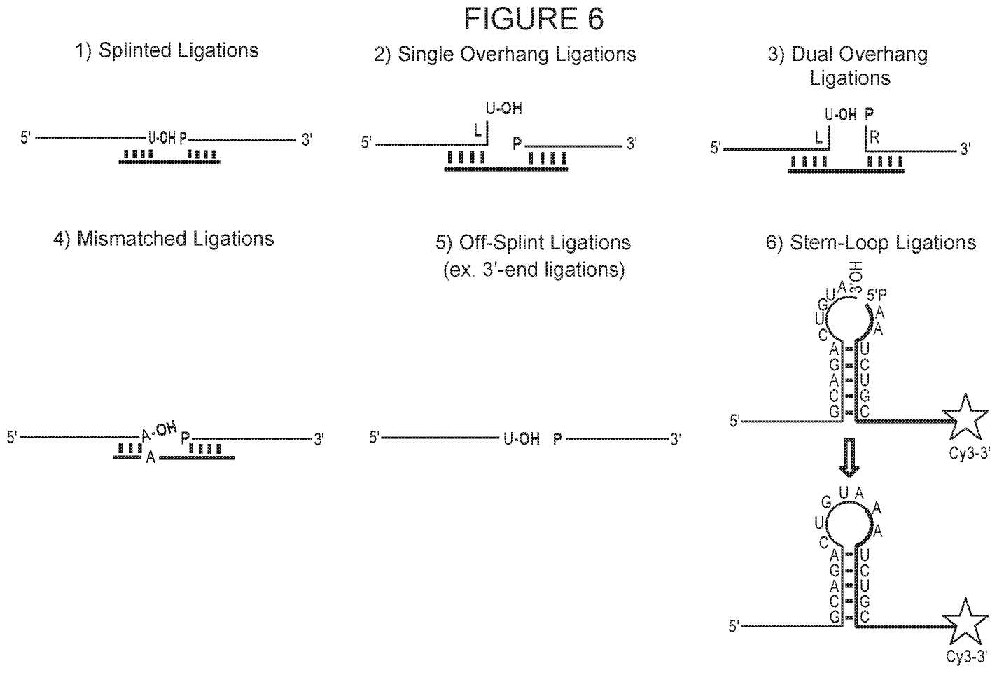

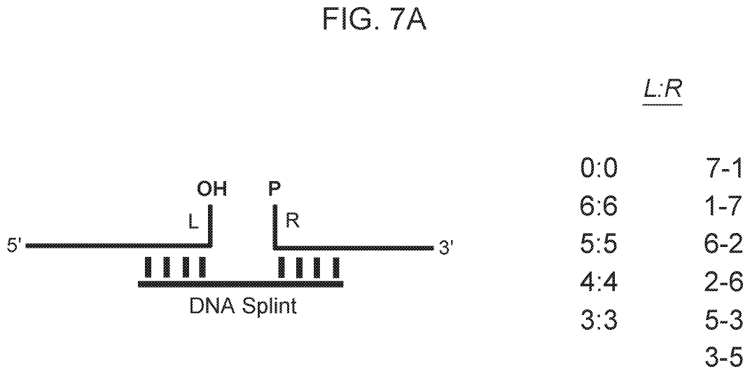

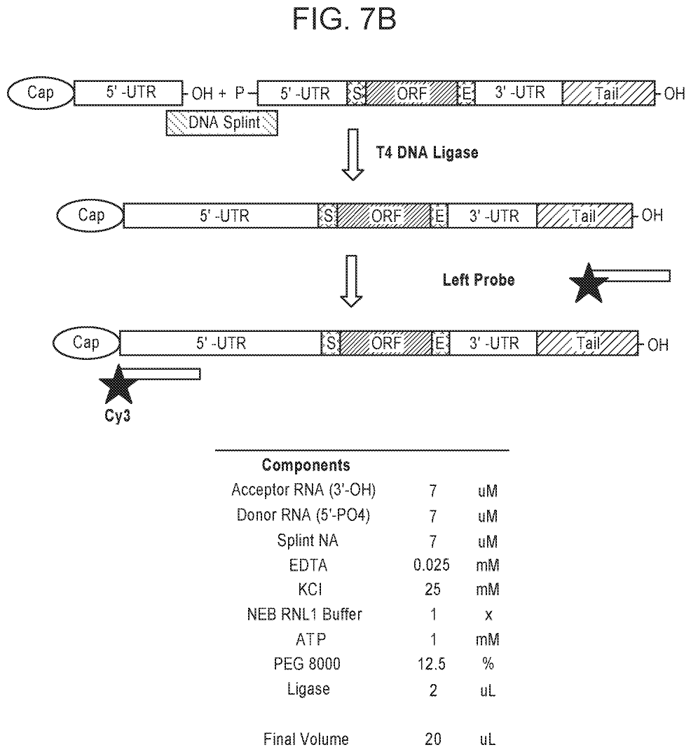

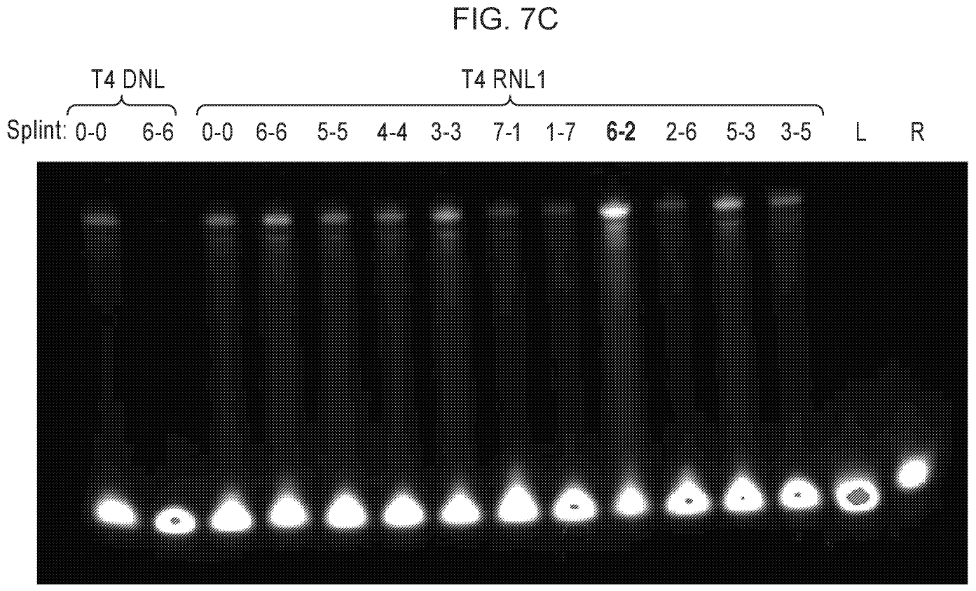

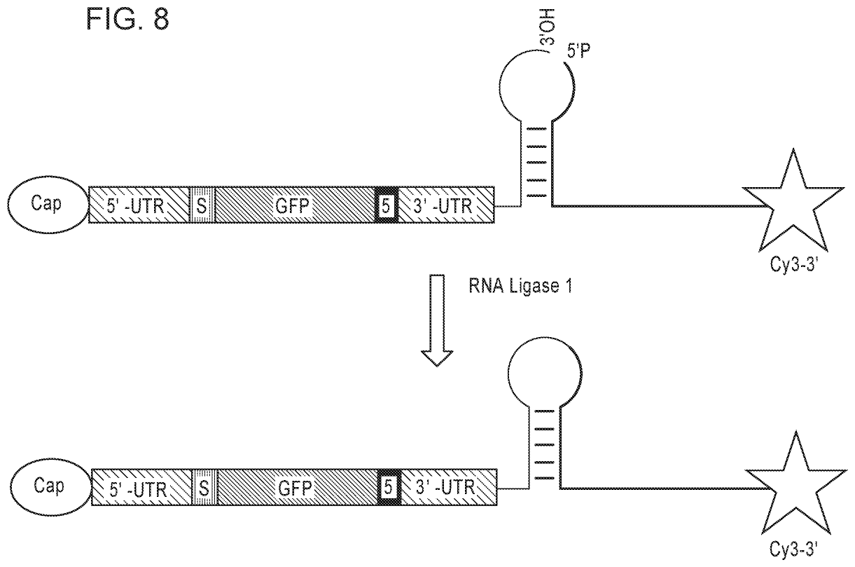

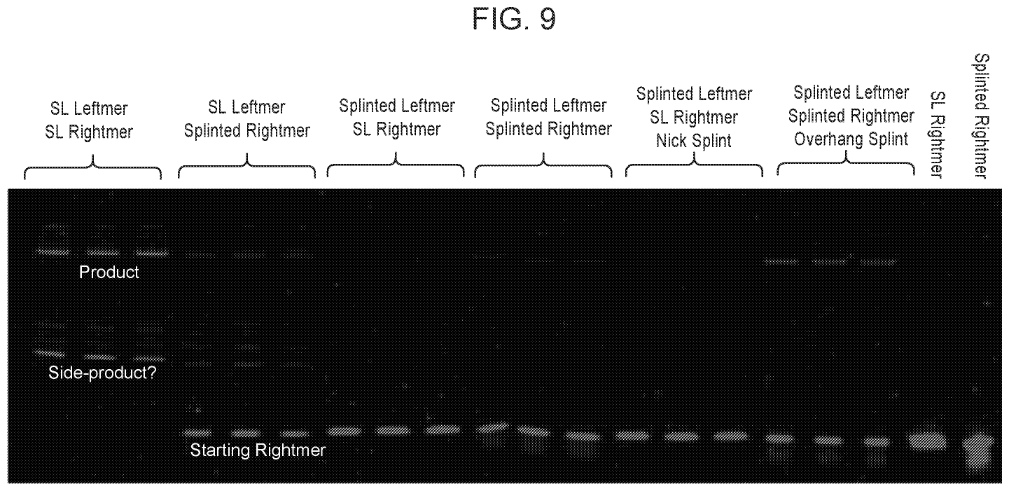

A ligation reaction as described in Example 2 was conducted on a 250 ug scale with a leftmer modified at various positions “A” (SEQ ID No. 4: ^{N7m}GpppGGGACUAGACUGAACUGGACA). The modification types and positions are summarized in FIG. 5A.

The reaction products were purified by reverse phase chromatography and the purified products were tested for expression in HEK293 cells (FIG. 5B).

### Example 4: Enzymatic Ligation as a Diagnostic for RNA 3′-End Homogeneity

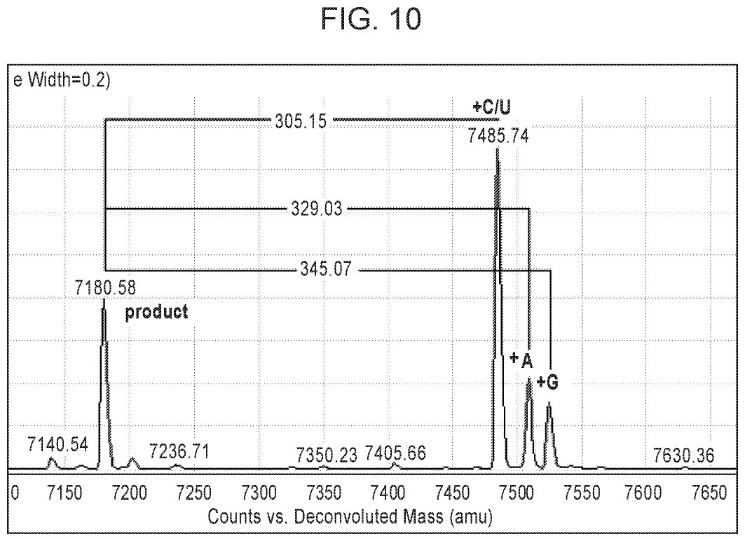

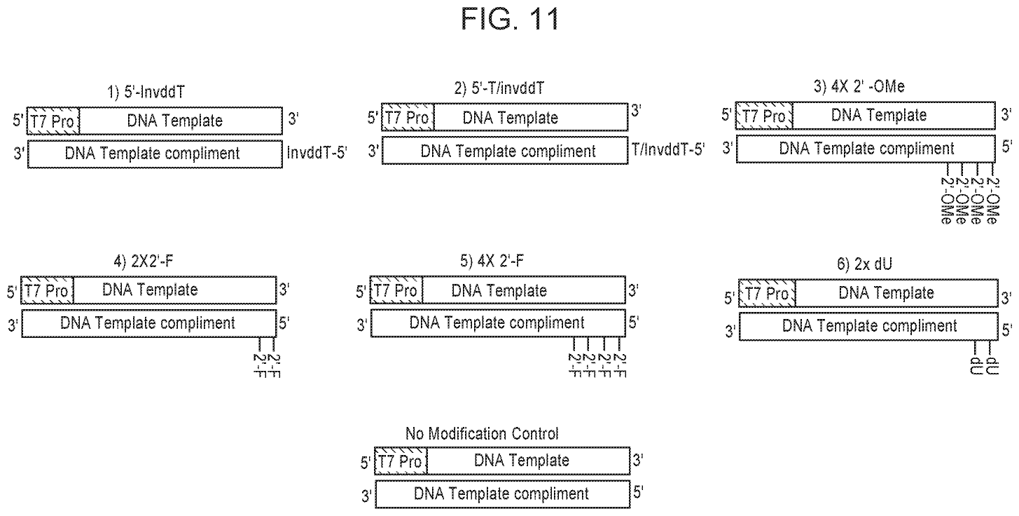

Certain properties of select enzymatic ligases are also useful for evaluating mRNA purity. T7 RNA Polymerase (T7RNP) is a near-ubiquitous RNA-dependent RNA polymerase. However, this enzyme can result in the untemplated addition of nucleotides to the 3′-end of RNA transcripts. It was found that T7RNP installed at least 1 but often 2 or more untemplated nucleotides at the 3′-end with seemingly little preference for nucleobase identity (FIG. 10). LC-MS traces of relatively short transcripts enabled the unambiguous determination that this activity was both general and very difficult to prevent using standard strategies such as the use of DNA templates with modifications at the 5′-end of the complement strand (FIG. 11).

Untemplated nucleotide addition at the 3′-end of mRNA is a significant problem for drug substance homogeneity and it is especially disruptive for strategies that rely upon downstream elaboration of the RNA by enzymatic ligation.

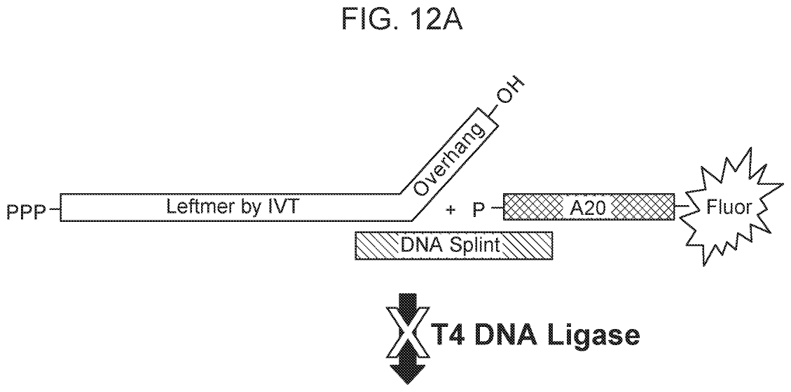

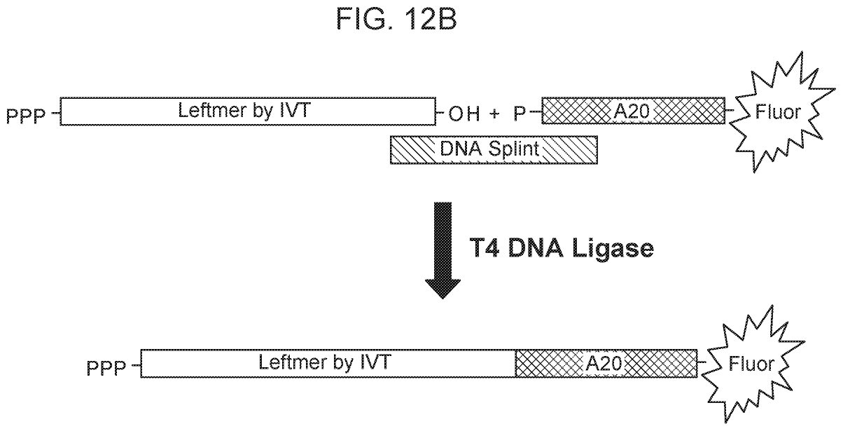

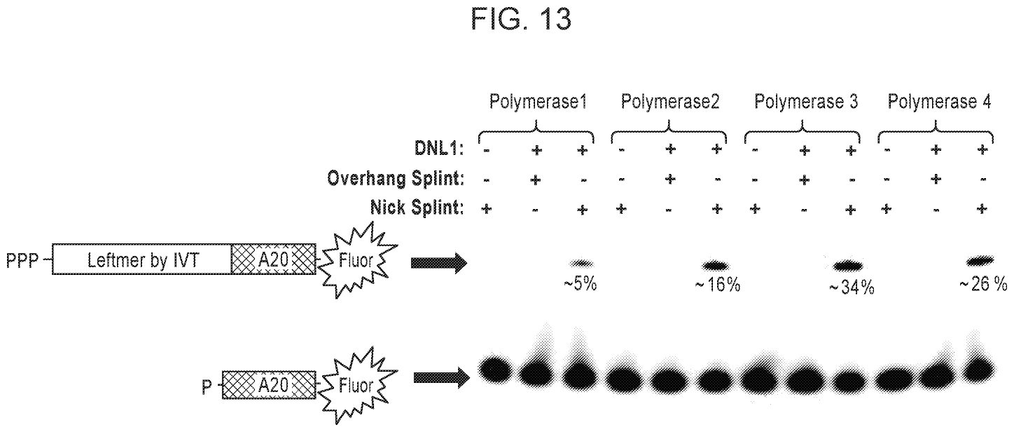

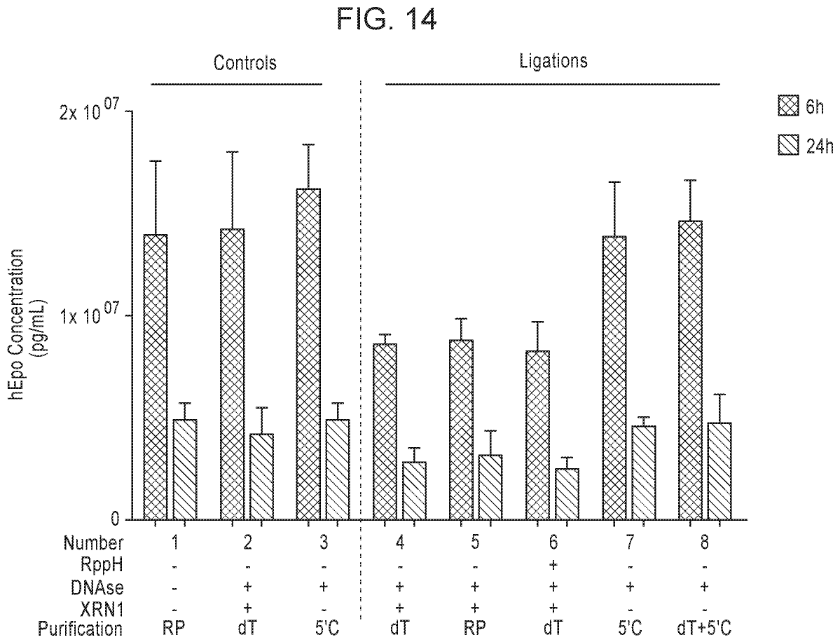

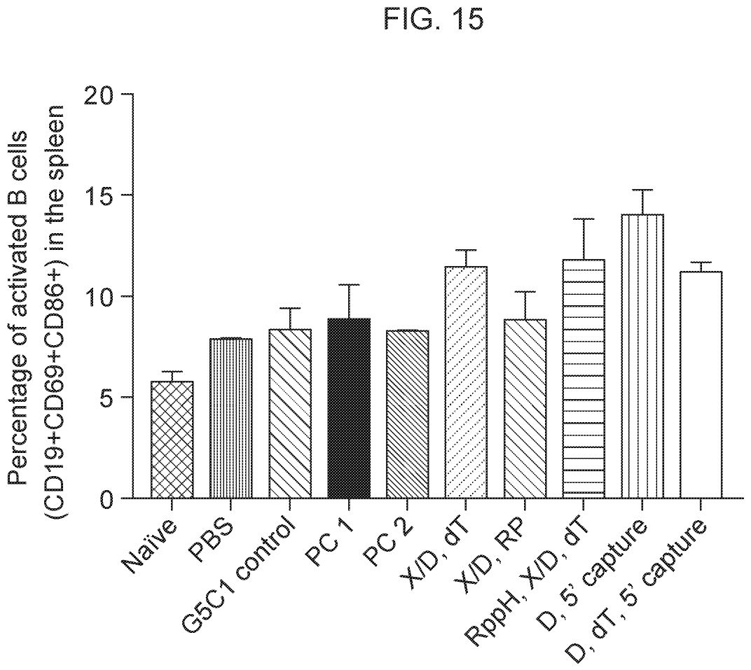

Enzymatic ligases differ in their substrate tolerance with regards to single stranded vs. double stranded nucleic acids, RNA vs. DNA, overhands vs. blunt ends, nicks vs. internal overhangs, etc. For example, T4 DNA Ligase 1 (DNL1), prefers substrates comprised of double-stranded DNA or RNA with a clean ‘nick’ at the site of ligation. Even a single nucleotide overhang at the ligation site predictably and entirely abolishes ligation efficiently (FIG. 12A). Thus, while ligation acceptors (i.e., leftmers) produced using T7RNP may be poor substrates for downstream elaboration in some ligations, this limitation also presents an opportunity in the form of a convenient and highly unambiguous assay for probing the extent of non-templated additions produced by variants of T7RNP or other polymerases. Ligation leftmers were made using an enzymatic polymerase. The leftmers were then annealed to a fully-complimentary DNA splint immediately adjacent to a 5′-monophosporylated rightmer RNA. The DNA splint was about 40 nt long and the rightmer was decorated with a fluorophore at the 5′-end. DNL1 was then added under catalytic conditions and ligation was allowed to proceed (FIG. 12B) for a desired reaction time before the reaction was quenched with EDTA. The mixture was then denatured in 4M urea at 95° C. for 5 minutes before being loaded onto a denaturing polyacrylamide gel (PAGE-D). The percentage of acrylamide in the gel was dependent on the length of the expected construct but was typically 6% acrylamide for leftmers >50 nt in length and 20% acrylamide for leftmers approaching the length of typical mRNA. 6% acrylamide gels were ran for 25 minutes at a constant 180V and 20% acrylamide gels were ran for 120 minutes at a constant 180V. The gels were then imaged on a biomolecular imager using excitation and emission wavelengths appropriate to the fluorophore present on the rightmer and the newly ligated product. Rightmer and ligated product should migrate differently on the gel based upon size and the intensity of the two bands are expected to exactly correspond to the efficiency of the ligation reaction. A greater extent of untemplated nucleotide addition during the enzymatic synthesis of the leftmer, is expected to lower the ligation yield (FIG. 13).

### EXEMPLARY EMBODIMENTS

### Embodiment 1

A method of preparing a modified RNA comprising enzymatically ligating an acceptor moiety having a 3′-hydroxyl group to the 5′-end of a donor moiety, wherein the donor moiety is an RNA comprising a leaving group.

### Embodiment 2

The method of Embodiment 1, wherein the enzymatic ligation is performed in a single enzymatic step.

### Embodiment 3

The method of Embodiment 1 or 2, wherein the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase, an ATP dependent DNA ligase, and combinations thereof.

### Embodiment 4

The method of Embodiment 3, wherein the thermostable DNA ligase is 5′AppDNA/RNA ligase.

### Embodiment 5

The method of Embodiment 3, wherein the ATP dependent DNA ligase is 9° N® DNA ligase.

### Embodiment 6

The method of Embodiment 3, wherein the ligation enzyme comprises T4 DNA ligase, T4 RNA ligase 1, T4 RNA ligase 2, T3 DNA ligase or T7 DNA ligase.

### Embodiment 7

The method of Embodiment 6, wherein the T4 RNA ligase 2 has a K227Q mutation.

### Embodiment 8

The method of Embodiment 6 or 7, wherein, the T4 RNA ligase 2 has a R55K mutation.

### Embodiment 9

The method of any one of Embodiments 6-8, wherein the T4 RNA ligase 2 is truncated.

### Embodiment 10

The method of Embodiment 6, wherein the ligation enzyme is T4 RNA ligase 1.

### Embodiment 11

The method of any one of the preceding Embodiments, wherein the donor moiety is an RNA comprising a non-naturally occurring nucleotide comprising one or more chemical modifications of a naturally occurring nucleotide.

### Embodiment 12

The method of Embodiment 11, wherein the nucleotide comprises a chemical modification located on the major groove face of the nucleobase portion of the nucleotide.

### Embodiment 13

The method of Embodiment 12, wherein the chemical modification comprises replacing an atom of the major groove face of the nucleobase with a group selected from optionally substituted amino, optionally substituted thiol, optionally substituted alkyl, optionally substituted alkenyl, and halo.

### Embodiment 14

The method of Embodiment 12, wherein the chemical modification comprises replacing an atom of the major groove face of the nucleobase with an amine, an SH, a methyl, an ethyl, a chloro or a fluoro group.

### Embodiment 15

The method of Embodiment 12 or 14, wherein the nucleobase portion comprises a pyrimidine nucleobase.

### Embodiment 16

The method of Embodiment 15, wherein the pyrimidine nucleobase is selected from the group consisting of cytosine (C), thymine (T), and uracil (U).

### Embodiment 17

The method of any one of the preceding Embodiments, wherein the nucleotide comprises a chemical modification located on the sugar.

### Embodiment 18

The method of Embodiment 17, wherein the modification is a modification at the 2′ position of the nucleoside.

### Embodiment 19

The method of Embodiment 18, wherein the chemical modification comprises 2′-O methylation.

### Embodiment 20

The method of Embodiment 17 or 18, wherein the chemical modification comprises replacing an atom of the sugar with a group selected from optionally substituted amino, optionally substituted thiol, optionally substituted azido, optionally substituted alkyl, optionally substituted alkenyl, and halo.

### Embodiment 21

The method of Embodiment 17 or 18, wherein the chemical modification comprises replacing an atom of the sugar with an amine, SH, N3, an alkyl, an alkenyl, or a halo group.

### Embodiment 22

The method of any one of Embodiments 17-21, wherein the chemical modification comprises a modification at the 4′-position of the nucleoside.

### Embodiment 23

The method of any one of the preceding Embodiments, wherein the donor moiety is an RNA comprising one or more chemical modifications located on the sugar-phosphate backbone.

### Embodiment 24

The method of Embodiment 23, wherein the chemical modification comprises replacing, one or more oxygens of the phosphodiester linkage with a group selected from optionally substituted amino, optionally substituted thiol, optionally substituted azido, optionally substituted alkyl, optionally substituted alkenyl, and halo.

### Embodiment 25

The method of Embodiment 23, wherein the chemical modification comprises replacing one or more oxygens of the phosphodiester linkage with an amine, S, or BH3.

### Embodiment 26

The method of any one of the preceding Embodiments, wherein one or more modifications are present in each of the sugar and the internucleotide linkage.

### Embodiment 27

The method of any one of the preceding Embodiments, wherein the donor moiety is an mRNA.

### Embodiment 28

The method of any one of the preceding Embodiments, wherein the 5′-end of the donor moiety comprises a 5′cap or 5′-cap analog.

### Embodiment 29

The method of any one of the preceding Embodiments, wherein the 5′-end of the donor moiety is a 5′-untranslated region.

### Embodiment 30

The method of any one of the preceding Embodiments, wherein the leaving group is a 5′-monophosphate group.

### Embodiment 31

The method of any one of Embodiments 1-29, wherein the leaving group is a 5′-AppN group.

### Embodiment 32

The method of any one of the preceding Embodiments, wherein the donor comprises a modified 3′-end.

### Embodiment 33

The method of Embodiment 32, wherein the modified 3′-end comprises modification that enhances purification, resistance to nucleases or ease of visualization.

### Embodiment 34

The method of Embodiment 33, wherein the modified 3′-end comprises a detectable agent.

### Embodiment 35

The method of Embodiment 33, wherein the modified 3′-end comprises a fluorophore.

### Embodiment 36

The method of Embodiment 35, wherein the fluorophore is selected from the group consisting of Cy3, Cy3.5, Cy5, Cy5.5, Cy7, GFP, and IR783.

### Embodiment 37

The method of any one of the preceding Embodiments, wherein the acceptor moiety comprises one or more nucleotides.

### Embodiment 38

The method of any one of the preceding Embodiments, wherein the acceptor moiety comprises between about two and about 850 nucleotides.

### Embodiment 39

The method of any one of the preceding Embodiments, wherein the acceptor moiety is selected from the group consisting of a dinucleotide, a trinucleotide, an mRNA cap, a cap-like structure and a non-cap like structure.

### Embodiment 40

The method any one of the preceding Embodiments, wherein the acceptor moiety is a dinucleotide.

### Embodiment 41

The method of Embodiment 40, wherein the dinucleotide comprises a 5′-triphosphate group or a 5′-inverted guanosine group.

### Embodiment 42

The method of any one of the preceding Embodiments, wherein the donor moiety comprises a chemically synthesized RNA.

### Embodiment 43

The method of any one of Embodiments 1-42, wherein the donor moiety comprises an enzymatically synthesized RNA.

### Embodiment 44

The method of any one of the preceding Embodiments, wherein the acceptor moiety further comprises an RNA.

### Embodiment 45

The method of Embodiment 44, wherein the enzymatic ligation further comprises the use of a single stranded DNA (ssDNA) splint.

### Embodiment 46

The method of Embodiment 45, wherein the ssDNA splint comprises a sequence complementary to at least one base pair at the 3′ end of the acceptor moiety, at least one basepair at the 5′ end of the donor moiety, or a combination thereof.

### Embodiment 47

The method of Embodiment 46, wherein the ssDNA splint comprises a DNA sequence complementary to between 1 and 20 basepairs at the 3′ end of the acceptor moiety.

### Embodiment 48

The method of Embodiment 46 or 47, wherein the ssDNA splint comprises a DNA sequence complementary to between 1 and 20 basepairs at the 5′ end of the donor moiety

### Embodiment 49

The method of Embodiment 46, wherein the ssDNA splint comprises a DNA sequence complementary to between 21 and 40 basepairs at the 3′ end of the acceptor moiety.

### Embodiment 50

The method of Embodiment 46 or 49, wherein the ssDNA splint comprises a DNA sequence complementary to between 21 and 40 basepairs at the 5′ end of the donor moiety

### Embodiment 51

The method of Embodiment 46, wherein the ssDNA splint comprises a DNA sequence complementary to at least 20 basepairs at the 3′ end of the acceptor moiety.

### Embodiment 52

The method of Embodiment 46 or 51, wherein the ssDNA splint comprises a DNA sequence complementary to between at least 20 basepairs at the 5′ end of the donor moiety

### Embodiment 53

The method of any one of Embodiments 46-52 wherein the length of the DNA sequence complementary to the 3′ end of the acceptor moiety and the length of the sequence complementary to the 5′ end of the donor moiety are not the same.

### Embodiment 54

The method of any one of Embodiments 46-52, wherein the length of the DNA sequence complementary to the 3′ end of the acceptor moiety and the length of the sequence complementary to the 5′ end of the donor moiety are the same.

### Embodiment 55

The method of any one of Embodiments 46-53, wherein the sequence complementary to the 3′ end of the acceptor moiety is offset from the 3′ terminus of the acceptor moiety by at least one basepair.

### Embodiment 56

The method of any one of Embodiments 46-54, wherein the sequence complementary to the 3′ end of the acceptor moiety comprises a sequence at least 2 nucleotides in length.

### Embodiment 57

The method of Embodiment 55, comprising at least one mismatch between the sequence of the ssDNA splint and the 3′ end of the acceptor moiety.

### Embodiment 58

The method of any one of Embodiments 1-44, wherein the 3′ end of the acceptor moiety and the 5′ end of the donor moiety form an RNA stem-loop.

### Embodiment 59

The method of any one of the preceding Embodiments, further comprising purification of the modified RNA

### Embodiment 60

The method of Embodiment 59, wherein the purifying comprises resolving the modified RNA from unreacted donor moiety.

### Embodiment 61

The method of Embodiment 60, wherein the resolving comprises enzymatically degrading the unreacted donor moiety.

### Embodiment 62

The method of Embodiment 61, wherein the enzyme is an exonuclease specific for 5′-monophosphate-containing RNA.

### Embodiment 63

The method of Embodiment 62, wherein the exonuclease is XRN-1.

### Embodiment 64

The method of any one of Embodiments 59-63, wherein the purification comprises separation of the modified RNA from the unreacted acceptor moiety.

### Embodiment 65

The method of Embodiment 64, wherein the separation comprises ultra-filtration.

### Embodiment 66

The method of Embodiment 59, wherein the purification comprises chromatographic methods.

### Embodiment 67

The method of Embodiment 59, wherein purification comprises affinity tag purification.

### Embodiment 68

The method of Embodiment 67, wherein the affinity tag comprises a chemical tag, an oligonucleotide or a 5′ cap.

### Embodiment 69

The method of Embodiment 68, wherein the chemical tag comprises biotin.

### Embodiment 70

The method of Embodiment 68, wherein the sequence of the oligonucleotide comprises a poly(A) sequence.

### Embodiment 71

The method of Embodiment 68, wherein the sequence of the oligonucleotide comprises a protein binding sequence.

### Embodiment 72

The method of Embodiment 71, wherein the protein binding sequence is an MS2 protein binding sequence.

### Embodiment 73

The method of Embodiment 68, wherein the sequence of the oligonucleotide comprises an aptamer.

### Embodiment 74

The method of Embodiment 73, wherein the aptamer binds to Streptavidin or Sephadex.

### Embodiment 75

The method of Embodiment 68, wherein the 5′ cap comprises a 5′ 7-methyl guanosine cap.

### Embodiment 76

The method of any of one of the preceding Embodiments, wherein the modified RNA product is a modified mRNA.

### Embodiment 77

A modified RNA product prepared by the method of any of one of the preceding Embodiments.

It is to be understood that while the compounds and methods of the present disclosure have been described in conjunction with the detailed description thereof, the foregoing description is intended to illustrate and not limit the scope of the present disclosure, which is defined by the scope of the appended claims. Other aspects, advantages, and alterations are within the scope of the following claims.

What is claimed is:

1. A method of preparing a modified RNA comprising enzymatically ligating an acceptor moiety having a 3′-hydroxyl group to the 5′-end of a donor moiety, wherein the donor moiety is an RNA comprising a leaving group.

2. The method of claim 1, wherein the enzymatic ligation is performed in a single enzymatic step.

3. The method of claim 1 or 2, wherein the ligation enzyme comprises T4 DNA ligase, T4 RNA ligase 1, T4 RNA ligase 2, T3 DNA ligase or T7 DNA ligase.

4. The method of claim 3, wherein the ligation enzyme is T4 RNA ligase 1.

5. The method of any one of the preceding claims, wherein the donor moiety is an RNA comprising a non-naturally occurring nucleotide comprising one or more chemical modifications of a naturally occurring nucleotide.

6. The method of claim 5, wherein the nucleotide comprises a chemical modification located on the major groove face of the nucleobase portion of the nucleotide.

7. The method of claim 6, wherein the chemical modification comprises replacing an atom of the major groove face of the nucleobase with an amine, an SH, a methyl, an ethyl, a chloro or a fluoro group.

8. The method of claim 6 or 7, wherein the nucleobase portion comprises a pyrimidine nucleobase.

9. The method of any one of the preceding claims, wherein the nucleotide comprises a chemical modification located on the sugar.

10. The method of claim 9, wherein the modification is a modification at the 2′ position of the nucleoside.

11. The method of claim 10, wherein the chemical modification comprises 2′-O methylation.

12. The method of claim 9 or 10, wherein the chemical modification comprises replacing an atom of the sugar with an amine, SH, N₃, an alkyl, an alkenyl, or a halo group.

13. The method of any one of claims 9-12, wherein the chemical modification comprises a modification at the 4′-position of the nucleoside.

14. The method of any one of the preceding claims, wherein the donor moiety is an RNA comprising one or more chemical modifications located on the sugar-phosphate backbone.

15. The method of claim 14, wherein the chemical modification comprises replacing one or more oxygens of the phosphodiester linkage with an amine, S, or BH₃.

16. The method of any one of the preceding claims, wherein one or more modifications are present in each of the sugar and the internucleotide linkage.

17. The method of any one of the preceding claims, wherein the donor moiety is an mRNA.

18. The method of any one of the preceding claims, wherein the 5′-end of the donor moiety comprises a 5′cap or 5′-cap analog.

19. The method of any one of the preceding claims, wherein the 5′-end of the donor moiety is a 5′-untranslated region.

20. The method of any one of the preceding claims, wherein the leaving group is a 5′-monophosphate group.

21. The method of any one of claims 1-19, wherein the leaving group is a 5′-AppN group.

22. The method of any one of the preceding claims, wherein the donor comprises a modified 3′-end.

23. The method of claim 22, wherein the modified 3′-end comprises modification that enhances purification, resistance to nucleases or ease of visualization.

24. The method of claim 23, wherein the modified 3′-end comprises a fluorophore.

25. The method of any one of the preceding claims, wherein the acceptor moiety comprises one or more nucleotides.

26. The method of any one of the preceding claims, wherein the acceptor moiety comprises between about two and about 850 nucleotides.

27. The method of any one of the preceding claims, wherein the acceptor moiety is selected from the group consisting of a dinucleotide, a trinucleotide, an mRNA cap, a cap-like structure, and a non-cap like structure.

28. The method any one of the preceding claims, wherein the acceptor moiety is a dinucleotide.

29. The method of claim 28, wherein the dinucleotide comprises a 5′-triphosphate group or a 5′-inverted guanosine group.

30. The method of any one of the preceding claims, wherein the donor moiety comprises a chemically synthesized RNA.

31. The method of any one of claims 1-30, wherein the donor moiety comprises an enzymatically synthesized RNA.

32. The method of any one of the preceding claims, wherein the acceptor moiety further comprises an RNA.

33. The method of claim 32, wherein the enzymatic ligation further comprises the use of a single stranded DNA (ssDNA) splint.

34. The method of claim 33, wherein the ssDNA splint comprises a sequence complementary to at least one base pair at the 3′ end of the acceptor moiety, at least one basepair at the 5′ end of the donor moiety, or a combination thereof.

35. The method of claim 34, wherein the ssDNA splint comprises a DNA sequence complementary to between 1 and 20 basepairs at the 3′ end of the acceptor moiety.

36. The method of claim 34 or 35, wherein the ssDNA splint comprises a DNA sequence complementary to between 1 and 20 basepairs at the 5′ end of the donor moiety.

37. The method of claim 34, wherein the ssDNA splint comprises a DNA sequence complementary to between 21 and 40 basepairs at the 3′ end of the acceptor moiety.

38. The method of claim 33 or 37, wherein the ssDNA splint comprises a DNA sequence complementary to between 21 and 40 basepairs at the 5′ end of the donor moiety.

39. The method of claim 34, wherein the ssDNA splint comprises a DNA sequence complementary to at least 20 basepairs at the 3′ end of the acceptor moiety.

40. The method of claim 34 or 39, wherein the ssDNA splint comprises a DNA sequence complementary to between at least 20 basepairs at the 5′ end of the donor moiety.

41. The method of any one of claims 33-35, wherein the length of the DNA sequence complementary to the 3′ end of the acceptor moiety and the length of the sequence complementary to the 5′ end of the donor moiety are not the same.

42. The method of any one of claims 33-35, wherein the length of the DNA sequence complementary to the 3′ end of the acceptor moiety and the length of the sequence complementary to the 5′ end of the donor moiety are the same.

43. The method of any one of claims 33-41, wherein the sequence complementary to the 3′ end of the acceptor moiety is offset from the 3′ terminus of the acceptor moiety by at least one basepair.

44. The method of any one of claims 33-42, wherein the sequence complementary to the 3′ end of the acceptor moiety comprises a sequence at least 2 nucleotides in length.

45. The method of claim 43, comprising at least one mismatch between the sequence of the ssDNA splint and the 3′ end of the acceptor moiety.

46. The method of any one of claims 1-32, wherein the 3′ end of the acceptor moiety and the 5′ end of the donor moiety form an RNA stem-loop.

47. The method of any one of the preceding claims, further comprising purification of the modified RNA.

48. The method of claim 47, wherein the purifying comprises resolving the modified RNA from unreacted donor moiety.

49. The method of claim 48, wherein the resolving comprises enzymatically degrading the unreacted donor moiety.

50. The method of claim 49, wherein the enzyme is an exonuclease specific for 5′-monophosphate-containing RNA.

51. The method of claim 50, wherein the exonuclease is XRN-1.

52. The method of any one of claims 47-51, wherein the purification comprises separation of the modified RNA from the unreacted acceptor moiety.

53. The method of claim 52, wherein the separation comprises ultra-filtration.

54. The method of claim 47, wherein the purification comprises chromatographic methods.

55. The method of claim 47, wherein purification comprises affinity tag purification.

56. The method of claim 55, wherein the affinity tag comprises a chemical tag, an oligonucleotide or a 5′ cap.

57. The method of claim 56, wherein the chemical tag comprises biotin.

58. The method of claim 56, wherein the sequence of the oligonucleotide comprises a poly(A) sequence.

59. The method of claim 56, wherein the sequence of the oligonucleotide comprises a protein binding sequence.

60. The method of claim 59, wherein the protein binding sequence is an MS2 protein binding sequence.

61. The method of claim 56, wherein the sequence of the oligonucleotide comprises an aptamer.

62. The method of claim 61, wherein the aptamer binds to Streptavidin or Sephadex.

63. The method of claim 56, wherein the 5′ cap comprises a 5′ 7-methyl guanosine cap.

64. The method of any of one of the preceding claims, wherein the modified RNA product is a modified mRNA.

65. A modified RNA product prepared by the method of any of one of the preceding claims.


SEQUENCE LISTING (6 sequences)

SEQ ID NO: 1 - DNA, 24 bp, Artificial Sequence
  chemically synthesized RNA
caaaggctcttttcagagccacca

SEQ ID NO: 2 - RNA, 24 nt, Artificial Sequence
  chemically synthesized RNA
caaaggcucuuuucagagccacca

SEQ ID NO: 3 - RNA, 26 nt, Artificial Sequence
  chemically synthesized RNA
  misc_feature (1)..(1): 5′-triphosphate
gaguaagaagaaauauaagagccacc

SEQ ID NO: 4 - RNA, 22 nt, Artificial Sequence
  chemically synthesized RNA
  misc_feature (1)..(1): 7-methylguanosine
  misc_feature (2)..(2): 5′-triphosphate
ggggacuagacugaacuggaca

SEQ ID NO: 5 - RNA, 10 nt, Artificial Sequence
  partial stem loop polynucleotide sequence
gcagacugua

SEQ ID NO: 6 - RNA, 17 nt, Artificial Sequence
  partial stem loop polynucleotide sequence
gcagacuguaaaucugc